(19) United States
(12) Patent Application Publication
(10) Pub. No.: US 2012/0010404 A1
(43) Pub. Date: Jan. 12, 2012

(54) OXAZINE DYES WITH IMPROVED AQUEOUS SOLUBILITY

(75) Inventor: Alexei Toutchkine, Arlington, MA (US)

(73) Assignee: SIGMA-ALDRICH CO., St. Louis, MO (US)

(21) Appl. No.: 12/993,121

(22) PCT Filed: Jun. 4, 2009
(86) PCT No.: PCT/US09/46238

§ 371 Date: Sep. 26, 2011

Related U.S. Application Data
(60) Provisional application No. 61/060,381, filed on Jun. 10, 2008.
(60) Provisional application No. 61/060,640, filed on Jun. 11, 2008.

(51) Int. Cl. C07D 498/14

(52) U.S. Cl. 544/99

(57) ABSTRACT

The present invention provides oxazine dyes, processes for preparing the oxazine dyes, intermediates that may be utilized in the processes for preparing the oxazine dyes, and methods for using the oxazine dyes.

### FIELD OF THE INVENTION

The invention generally provides dye compounds, intermediates that can be used in processes to synthesize the dye compounds, and methods for synthesizing the dye compounds. More particularly, the dye compounds are oxazine dyes.

### BACKGROUND OF THE INVENTION

Long wavelength light-emitting (i.e., 600-1000 nm) fluorescent chromophores are useful in a variety of biological applications because, in this region of the spectrum, there is minimum interference from the natural autofluorescence of biological molecules, as well as reduced cell damage (because of decreased absorption scattering). Among the long wavelength emitting dyes, the oxazine derivatives offer significant advantages over rhodamine, squarine, and cyanine dyes. Oxazine derivatives not only exhibit strong fluorescence, but also have excellent chemical and photochemical stability. One disadvantage of oxazine-based dyes in biological applications, however, is their low solubility in water and their tendency to aggregate in aqueous solutions.

Attempts to increase the water solubility of oxazines have been met with limited success, however. Generally, modifications to oxazine-derived molecules have negatively affected the chemical and/or the spectral properties of the dyes. There is a need, therefore, for oxazine derivatives, and methods of making, that have a high solubility in water and that also display excellent chemical stability, photostability, and strong fluorescence.

### SUMMARY OF THE INVENTION

Among the various aspects of the present invention is the provision of oxazine dye compounds and methods of making the dye compounds.

A first aspect of the invention encompasses a compound comprising Formula (I):

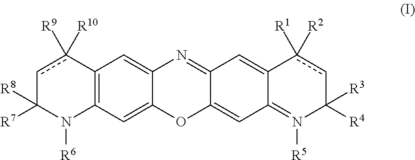

wherein:
R¹, R², R⁹, and R¹⁰ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide;
R³, R⁴, R⁷, and R⁸ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide; provided that R³ and R⁴ together and/or R⁷ and R⁸ together may form a saturated and/or unsaturated bridge;
R⁵ and R⁶ are independently selected from the group consisting of {—}L₁-Rₓ, hydrogen, hydrocarbyl, substituted hydrocarbyl, and a group that has a positive charge or negative charge at a pH from about 6 to about 8; provided that at least one of R⁵ or R⁶ comprises a group that has a positive charge or negative charge at a pH from about 6 to about 8; provided that if one of R⁵ or R⁶ is an alkyl group then the other of R⁵ or R⁶ does not comprise a carboxylate group;
L₁ is a linker;
Rₓ is a group selected from the group consisting of phosphate, sulfonate, carboxylic acid, carboxylic ester, isocyanate, isothiocyanate, maleimide, haloacetamide, and alkyl halide; and
-------- is a double or single bond; provided that when a double bond is present then R² and/or R¹⁰ are not present; and provided that when R¹ is hydrogen and R² is SO₃H, then at least one of R⁷ or R⁸ is not hydrogen.

Another aspect of the present provides a compound comprising Formula (II):

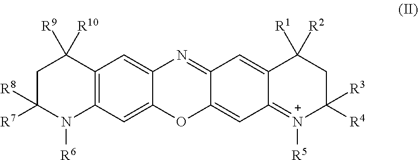

wherein:
R¹, R², R⁹, and R¹⁰ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide;
R³, R⁴, R⁷, and R⁸ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide; provided that R³ and R⁴ together and/or R⁷ and R⁸ together may form a saturated and/or unsaturated bridge;
R⁵ and R⁶ are independently selected from the group consisting of {—}L₁-Rₓ, hydrogen, hydrocarbyl, substituted hydrocarbyl, and a group that has a positive charge or negative charge at a pH from about 6 to about 8; provided that at least one of R⁵ or R⁶ comprises a group that has a positive charge or negative charge at a pH from about 6 to about 8; provided that if one of R⁵ or R⁶ is an alkyl group then the other of R⁵ or R⁶ does not comprise a carboxylate group;
L₁ is a linker;
Rₓ is a group selected from the group consisting of phosphate, sulfonate, carboxylic acid, carboxylic ester, isocyanate, isothiocyanate, maleimide, haloacetamide, and alkyl halide; and
provided that when R¹ is hydrogen and R² is SO₃H, then at least one of R⁷ or R⁸ is not hydrogen.

A further aspect of the invention encompasses compound comprising Formula (III):

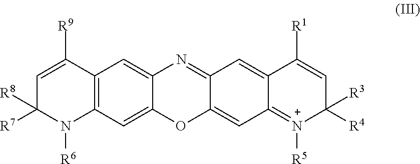

wherein:
R¹ and R⁹ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide;
R³, R⁴, R⁷, and R⁸ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide; provided that R³ and R⁴ together and/or R⁷ and R⁸ together may form a saturated and/or unsaturated bridge; provided that when R¹ is SO₃H, then at least one of R⁷ or R⁸ is not hydrogen;
R⁵ and R⁶ are independently selected from the group consisting of {—}L₁-Rₓ, hydrogen, hydrocarbyl, substituted hydrocarbyl, and a group that has a positive charge or negative charge at a pH from about 6 to about 8; provided that at least one of R⁵ or R⁶ comprises a group that has a positive charge or negative charge at a pH from about 6 to about 8; provided that if one of R⁵ or R⁶ is an alkyl group then the other of R⁵ or R⁶ does not comprise a carboxylate group;

L₁ is a linker; and

Rₓ is a group selected from the group consisting of phosphate, sulfonate, carboxylic acid, carboxylic ester, isocyanate, isothiocyanate, maleimide, haloacetamide, and alkyl halide.

Still another aspect of the present invention provides a compound comprising Formula (V):

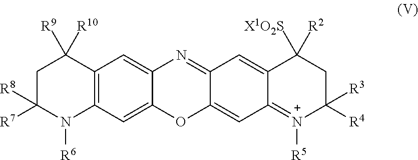

wherein:
R², R⁹, and R¹⁰ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide;
R³, R⁴, R⁷, and R⁸ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide; provided that R³ and R⁴ together and/or R⁷ and R⁸ together may form a saturated and/or unsaturated bridge;
R⁵ and R⁶ are independently selected from the group consisting of {—}L₁-Rₓ, hydrogen, hydrocarbyl, substituted hydrocarbyl, and a group that has a positive charge or negative charge at a pH from about 6 to about 8; provided that at least one of R⁵ or R⁶ comprises a group that has a positive charge or negative charge at a pH from about 6 to about 8; provided that if one of R⁵ or R⁶ is an alkyl group then the other of R⁵ or R⁶ does not comprise a carboxylate group;
L₁ is a linker;
Rₓ is a group selected from the group consisting of carboxylic acid, carboxylic ester, isocyanate, isothiocyanate, maleimide, haloacetamide, and alkyl halide; and
X¹ is selected from the group consisting of O⁻, ONa, OLi, OK, ONH₄, and ONR₄, wherein R is an alkyl group.

A further aspect of the invention encompasses a compound comprising Formula (VI):

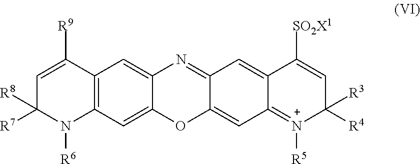

wherein:
R³, R⁴, R⁷, and R⁸ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide; provided that R³ and R⁴ together and/or R⁷ and R⁸ together may form a saturated and/or unsaturated bridge;
R⁵ and R⁶ are independently selected from the group consisting of {—}L₁-Rₓ, hydrogen, hydrocarbyl, substituted hydrocarbyl, and a group that has a positive charge or negative charge at a pH from about 6 to about 8; provided that at least one of R⁵ or R⁶ comprises a group that has a positive charge or negative charge at a pH from about 6 to about 8; provided that if one of R⁵ or R⁶ is an alkyl group then the other of R⁵ or R⁶ does not comprise a carboxylate group;
R⁹ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide;
L₁ is a linker;
Rₓ is a group selected from the group consisting of carboxylic acid, carboxylic ester, isocyanate, isothiocyanate, maleimide, haloacetamide, and alkyl halide; and
X¹ is selected from the group consisting of O⁻, ONa, OLi, OK, ONH₄, and ONR₄, wherein R is an alkyl group.

Still another aspect of the invention provides a process for preparing a compound comprising Formula (II). The process comprises contacting a compound comprising Formula (i) with a compound comprising Formula (ii) in the presence of a proton donor to form the compound comprising Formula (II):

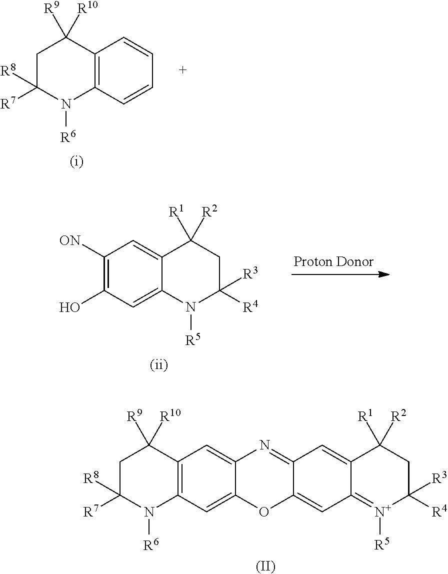

wherein:
R¹, R², R⁹, and R¹⁰ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide;
R³, R⁴, R⁷, and R⁸ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide; provided that R³ and R⁴ together and/or R⁷ and R⁸ together may form a saturated and/or unsaturated bridge;
R⁵ and R⁶ are independently selected from the group consisting of {—}L₁-Rₓ, hydrogen, hydrocarbyl, substituted hydrocarbyl, and a group that has a positive charge or negative charge at a pH from about 6 to about 8; provided that at least one of R⁵ or R⁶ comprises a group that has a positive charge or negative charge at a pH from about 6 to about 8; provided that if one of R⁵ or R⁶ is an alkyl group then the other of R⁵ or R⁶ does not comprise a carboxylate group; and
the proton donor has a pKa less than about 6.

A further aspect of the invention encompasses a process for preparing a compound comprising Formula (II). The process comprises contacting a compound comprising Formula (iii) with a compound comprising Formula (iv) in the presence of a proton donor to form the compound comprising Formula (II):

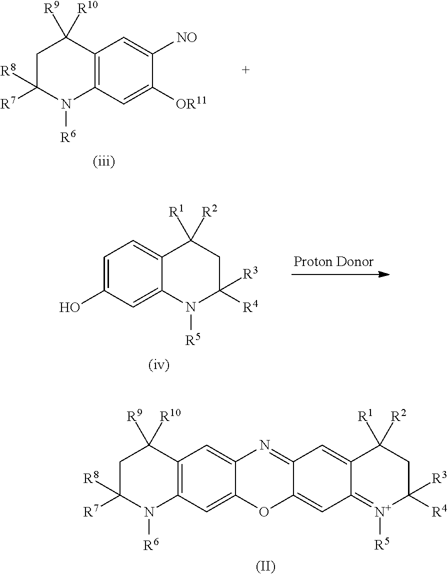

wherein:
R¹, R², R⁹, and R¹⁰ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide;
R³, R⁴, R⁷, and R⁸ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide; provided that R³ and R⁴ together and/or R⁷ and R⁸ together may form a saturated and/or unsaturated bridge;
R⁵ and R⁶ are independently selected from the group consisting of {—}L₁-Rₓ, hydrogen, hydrocarbyl, substituted hydrocarbyl, and a group that has a positive charge or negative charge at a pH from about 6 to about 8; provided that at least one of R⁵ or R⁶ comprises a group that has a positive charge or negative charge at a pH from about 6 to about 8; provided that if one of R⁵ or R⁶ is an alkyl group then the other of R⁵ or R⁶ does not comprise a carboxylate group;
R¹¹ is selected from the group consisting of hydrogen and alkyl; and
the proton donor has a pKa less than about 6.

Still another aspect of the invention provides a process for preparing a compound comprising Formula (II). The process comprises contacting a compound comprising Formula (v) with a compound comprising Formula (iv) in the presence of a proton donor to form the compound comprising Formula (II):

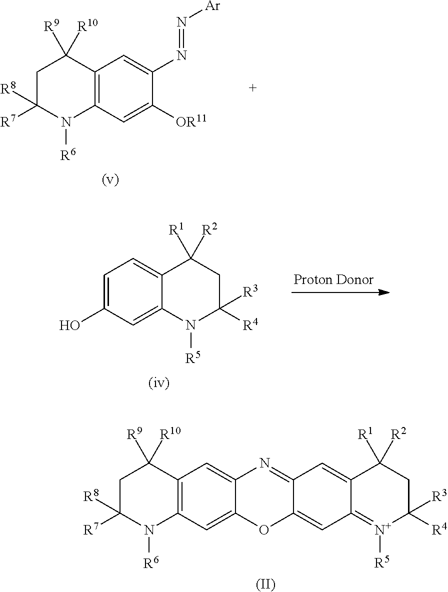

wherein:
R¹, R², R⁹, and R¹⁰ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide;
R³, R⁴, R⁷, and R⁸ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide; provided that R³ and R⁴ together and/or R⁷ and R⁸ together may form a saturated and/or unsaturated bridge;
R⁵ and R⁶ are independently selected from the group consisting of {—}L₁-Rₓ, hydrogen, hydrocarbyl, substituted hydrocarbyl, and a group that has a positive charge or negative charge at a pH from about 6 to about 8; provided that at least one of R⁵ or R⁶ comprises a group that has a positive charge or negative charge at a pH from about 6 to about 8; provided that if one of R⁵ or R⁶ is an alkyl group then the other of R⁵ or R⁶ does not comprise a carboxylate group;
R¹¹ is selected from the group consisting of hydrogen and alkyl;
Ar comprises C₆H₄Xₐ;
Xₐ comprises an electron withdrawing group; and
the proton donor has a pKa less than about 6.

Other aspects and features of the invention are described in more detail below.

### DESCRIPTION OF THE FIGURES

FIG. 1 presents a comparison of the absorbance of Dye 3/IgG and dye Mr121/IgG conjugates. A. Absorbance and excitation spectra of goat anti-mouse IgG labeled with oxazine dye Mr121, degree of labeling (DOL)=4. B. Absorbance and excitation of goat anti-mouse IgG labeled with Dye 3, DOL=5. All concentrations are equal to 5 μM.

FIG. 2 illustrates that the total fluorescence of the dye/IgG conjugates depends on the degree of labeling. Plotted is the total fluorescent of Dye 3/IgG (SD3-GAM) and Dye 14/IgG (SD14/GAM) conjugates as a function of the dye to protein ration. All concentrations are equal to 1 μM.

FIG. 3 shows the photobleaching of dye-protein conjugates. Goat anti-mouse IgG was labeled with Dye 14, DyLight 680, Alexa 660, or Alexa 680 dyes. Plotted is the normalized fluorescence as a function of time.

FIG. 4 illustrates the fluorescence quenching by tryptophan. Shown is the fluorescence of Dye 3 in the absence and presence of various concentration of tryptophan (Trp) in phosphate buffer (pH 8) as a function of wavelength.

### DETAILED DESCRIPTION OF THE INVENTION

The present invention provides oxazine dyes, intermediate compounds that may be utilized in processes for making the oxazine dyes, and methods for using the oxazine dyes. Advantageously, the oxazine dyes of the invention have been engineered to improve aqueous solubility, while retaining excellent fluorescence. The oxazine dyes, when conjugated to an analyte (e.g., peptide, protein, or nucleic acid), also improve the aqueous solubility of the analyte. Because of these advantageous properties, the oxazine dyes may be utilized to assay, determine, measure, detect, visualize, and locate a wide array of analytes in cells, biological fluids, other biological systems, and other media.

(I) Oxazine Dye Compounds

One aspect of the invention provides dye compounds that comprise Formula (I):

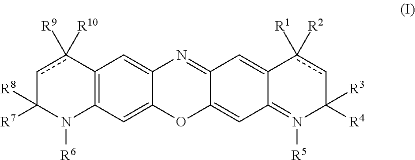

wherein:
R¹, R², R⁹, and R¹⁰ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide;
R³, R⁴, R⁷, and R⁸ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide; provided that R³ and R⁴ together and/or R⁷ and R⁸ together may form a saturated and/or unsaturated bridge;
R⁵ and R⁶ are independently selected from the group consisting of {—}L₁-Rₓ, hydrogen, hydrocarbyl, substituted hydrocarbyl, and a group that has a positive charge or negative charge at a pH from about 6 to about 8; provided that at least one of R⁵ or R⁶ comprises a group that has a positive charge or negative charge at a pH from about 6 to about 8; provided that if one of R⁵ or R⁶ is an alkyl group then the other of R⁵ or R⁶ does not comprise a carboxylate group;
L₁ is a linker;
Rₓ is a group selected from the group consisting of phosphate, sulfonate, carboxylic acid, carboxylic ester, isocyanate, isothiocyanate, maleimide, haloacetamide, and alkyl halide; and
-------- is a double or single bond; provided that when a double bond is present then R² and/or R¹⁰ are not present; and provided that when R¹ is hydrogen and R² is SO₃H, then at least one of R⁷ or R⁸ is not hydrogen.

In one exemplary embodiment, the hydrocarbyl and/or substituted hydrocarbyl is selected from the group consisting of alkyl, cycloalkyl, alkenyl, alkenoxy, aryl, alkylaryl, arylalkoxyl, alkoxy, alkoxycarbonyl, carbonyl, acyl, acyloxy, sulfonyl, sulfonyl halide, sulfonyl ester, carboxyl, and carboxylic acid. In one alternative of this embodiment, at least one of the carbon atoms forming the hydrocarbyl or substituted hydrocarbyl is substituted with a group selected from the group consisting of hydroxyl, halogen, cyano, nitro, carbonyl, acyl, acyloxy, carboxyl, phosphate, sulfonyl, sulfonyl halide, carboxylic acid ester, sulfonyl ester, amino, amido, azide, thiocyanate, isothiocyanate, and imide. In other alternative embodiments, at least two of, at least three of, or more than four of the carbon atoms forming the hydrocarbyl or substituted hydrocarbyl may be substituted with any of the aforementioned groups.

To increase the aqueous solubility of the dye compound, at least one of R⁵ or R⁶ is an aqueous solubilizing group that is attached to a nitrogen ring atom via a linker. In the context of the invention, the term “aqueous-solubilizing group” is used in its broadest sense to include groups that improve the overall solubility of the compound having Formula (I) when it is present in an aqueous solution relative to the solubility of the compound if the aqueous solubilizing group wasn't attached. In certain iterations of the invention, both R⁵ and R⁶ comprise an aqueous solubilizing group. The aqueous solubilizing group generally comprises a group that has a positive charge or negative charge at a pH from about 6 to about 8. In an exemplary iteration, the aqueous solubilizing group comprises L₂-X. L₂ is a linker that attaches X to a nitrogen ring atom, and X is selected from the group consisting of sulfate, sulfanate, sulfinate, phosphate, phosphonate, phosphinate, carboxylate, amine, alkyl amine, dialkylamine, and an ammonium salt.

In one alternative embodiment, at least one of R¹, R², R³, R⁴, R⁵, R⁶, R⁷, R⁸, R⁹, and R¹⁰ comprises a reactive group that is attached to a carbon or nitrogen ring atom by a linker. The reactive group may be utilized to covalently conjugate the dye compound to an analyte, such as a protein or a nucleic acid. In this context, the reactive group attached to the dye compound is selected so that it reacts with a functional group on the analyte to form a covalent bond that conjugates the dye compound to the analyte. In an exemplary embodiment, the reactive group and linker comprise L₁-Rₓ. L₁ is a linker that attaches the reactive group, Rₓ, to a carbon or nitrogen ring atom. Non-limiting examples of suitable Rₓ groups along with suitable functional groups (disposed on the analyte), and the resulting covalent linkage that conjugates the dye compound to the analyte are depicted in Table 1.

TABLE 1

| | Functional | Resulting Covalent |
|---|---|---|
| Reactive Group | Group | Linkage |
| activated carboxylic ester | amine | carboxyamide |
| isothiocyanate | amine | thiourea |
| isocyanate | amine | urea |
| carboxylic acid | amine | carboxyamide |
| carboxylic acid | hydrazine | hydrazide |
| maleimide | thiol | thioether |
| haloacetamide | thiol | thioether |
| alkyl halide | amine | alkylamine |
| alkyl halide | thiol | thioether |

In some embodiments, at least two of R¹, R², R³, R⁴, R⁵, R⁶, R⁷, R⁸, R⁹, and R¹⁹ are {—}L₁-Rₓ. In an alternative embodiment, at least three of R¹, R², R³, R⁴, R⁵, R⁶, R⁷, R⁸, R⁹, and R¹⁹ are {—}L₁-Rₓ. In an additional embodiment, three or more of R¹, R², R³, R⁴, R⁵, R⁶, R⁷, R⁸, R⁹, and R¹⁹ are {—}L₁-Rₓ.

The chain of atoms defining the linker, L₁ or L₂, can and will vary depending upon the embodiment. The linker may comprise a variety of carbon or heteroatoms that may be saturated or unsaturated, substituted or unsubstituted, linear or cyclic, or straight or branched. The chain of atoms defining the linker will typically be selected from a hydrocarbyl or a substituted hydrocarbyl. For example, the atoms forming the linker may be selected from the group consisting of carbon, oxygen, nitrogen, sulfur, selenium, silicon and phosphorous. In one exemplary embodiment, the linker may be selected from the group consisting of alkyl groups, alkenyl groups, aryl groups, and heteroaryl groups. The alkyl groups, alkenyl groups, aryl groups, and heteroaryl groups may be substituted with at least one heteroatom. It will also be appreciated that the number of atoms forming the linker can and will vary. In one embodiment, the number of atoms forming the linker may range from about 1 to about 30. In an alternative embodiment, the number of atoms forming the linker may range from about 2 to about 10.

In an exemplary embodiment for compounds having Formula (I), R³, R⁴, R⁷, and R⁸ are independently selected from the group consisting of hydrogen and an alkyl group. In one alternative of this embodiment, R³, R⁴, R⁷, and R⁸ are independently selected from the group consisting of hydrogen and methyl.

In one alternative embodiment, the compound may comprise Formula (II):

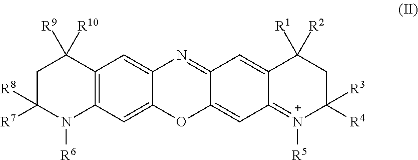

wherein:
R¹, R², R³, R⁴, R⁵, R⁶, R⁷, R⁸, R⁹, R¹⁰, Rₓ and L₁ are as described for compounds having Formula (I).

In one exemplary embodiment for compounds having Formula (II), R³, R⁴, R⁷, and R⁸ are independently selected from the group consisting of hydrogen and an alkyl group. In an alternative of this embodiment, R³, R⁴, R⁷, and R⁸ are independently selected from the group consisting of hydrogen and methyl. In another alternative of this embodiment, R³, R⁴, R⁷, and R⁸ are each methyl. In still another alternative of this embodiment, R³, and R⁴ are each methyl; and R⁷, and R⁸ are each hydrogen. In an additional alternative of this embodiment, R³, and R⁴ are each hydrogen; and R⁷ and R⁸ are each methyl.

In another exemplary embodiment for compounds having Formula (II), one of R¹ or R² is SO₂X¹. Exemplary X¹ groups include O⁻, ONa, OLi, OK, ONH₄, and ONR₄, wherein R is an alkyl group. In an additional aspect of this embodiment, R³, R⁴, R⁷, and R⁸ are independently selected from the group consisting of hydrogen and methyl.

In an additional exemplary embodiment for compounds having Formula (II), R⁵ and R⁶ are independently selected from the group consisting of {—}(CH₂)ₘCOX² and {—}(CH₂)ₘSO₃⁻; m is an integer from 1 to 5; X² is selected from the group consisting of hydroxyl, a N-succinimidyl ester group, and a 2-(N-maleimidoethyl)amino group; and one of R⁹ and R¹⁰ is hydrogen, and the other of R⁹ and R¹⁰ is selected from the group consisting of hydrogen, methyl, and {—}(CH₂)ₘSO₃⁻.

In one alternative embodiment, the compound may comprise Formula (III):

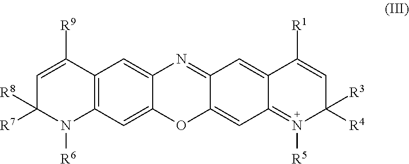

wherein:
R¹, R³, R⁴, R⁵, R⁶, R⁷, R⁸, R⁹, Rₓ and L₁ are as described for compounds having Formula (I).

In one exemplary embodiment for compounds having Formula (III), R³, R⁴, R⁷, and R⁸ are independently selected from the group consisting of hydrogen and an alkyl group. In an alternative of this embodiment, R³, R⁴, R⁷, and R⁸ are independently selected from the group consisting of hydrogen and methyl. In another alternative of this embodiment, R³, R⁴, R⁷, and R⁸ are each methyl. In still another alternative of this embodiment, R³, and R⁴ are each methyl; and R⁷, and R⁸ are each hydrogen. In an additional alternative of this embodiment, R³, and R⁴ are each hydrogen; and R⁷ and R⁸ are each methyl.

In another exemplary embodiment for compounds having Formula (III), one of R¹ or R⁹ is SO₂X¹. Exemplary X¹ groups include O⁻, ONa, OLi, OK, ONH₄, and ONR₄, wherein R is an alkyl group. In an additional aspect of this embodiment, R³, R⁴, R⁷, and R⁸ are independently selected from the group consisting of hydrogen and methyl.

In an additional exemplary embodiment for compounds having Formula (III), R⁵ and R⁶ are independently selected from the group consisting of {—}(CH₂)ₘCOX² and {—}(CH₂)ₘSO₃⁻, wherein m is an integer from 1 to 5; and X² is selected from the group consisting of hydroxyl, a N-succinimidyl ester group, and a 2-(N-maleimidoethyl)amino group.

In one alternative embodiment, the compound may comprise Formula (IV):

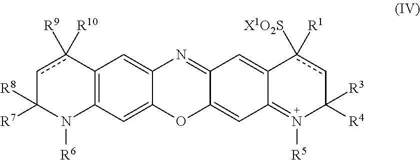

wherein:
R², R³, R⁴, R⁵, R⁶ R⁷, R⁸, R⁹, R¹⁰, Rₓ, --------, and L₁ are as described for compounds having Formula (I), and X¹ is selected from the group consisting of O⁻, ONa, OLi, OK, ONH₄, and ONR₄, wherein R is an alkyl group.

In one exemplary embodiment for compounds having Formula (IV), R³, R⁴, R⁷, and R⁸ are independently selected from the group consisting of hydrogen and an alkyl group. In an alternative of this embodiment, R³, R⁴, R⁷, and R⁸ are independently selected from the group consisting of hydrogen and methyl. In another alternative of this embodiment, R³, R⁴, R⁷, and R⁸ are each methyl. In still another alternative of this embodiment, R³ and R⁴ are each methyl; and R⁷ and R⁸ are each hydrogen. In an additional alternative of this embodiment, R³ and R⁴ are each hydrogen; and R⁷ and R⁸ are each methyl.

In yet another embodiment, the compound comprises Formula (V):

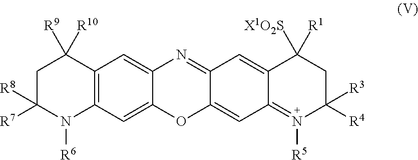

wherein:
R², R³, R⁴, R⁵, R⁶, R⁷, R⁸, R⁹, R¹⁰, Rₓ, and L₁ are as described for compounds having Formula (I), and X¹ is selected from the group consisting of O⁻, ONa, OLi, OK, ONH₄, and ONR₄, wherein R is an alkyl group.

In one exemplary embodiment for compounds having Formula (V), R³, R⁴, R⁷, and R⁸ are independently selected from the group consisting of hydrogen and an alkyl group. In an alternative of this embodiment, R³, R⁴, R⁷, and R⁸ are independently selected from the group consisting of hydrogen and methyl. In another alternative of this embodiment, R³, R⁴, R⁷, and R⁸ are each methyl. In still another alternative of this embodiment, R³, and R⁴ are each methyl; and R⁷ and R⁸ are each hydrogen. In an additional alternative of this embodiment, R³ and R⁴ are each hydrogen; and R⁷ and R⁸ are each methyl.

In an additional exemplary embodiment for compounds having Formula (V), R⁵ and R⁶ are independently selected from the group consisting of {—}(CH₂)ₘCOX² and {—}(CH₂)ₘSO₃⁻; m is an integer from 1 to 5; X² is selected from the group consisting of hydroxyl, a N-succinimidyl ester group, and a 2-(N-maleimidoethyl)amino group; and one of R⁹ and R¹⁰ is hydrogen, and the other of R⁹ and R¹⁰ is selected from the group consisting of hydrogen, methyl, and {—}(CH₂)ₘSO₃⁻. In one alternative of this embodiment, R³, R⁴, R⁷, and R⁸ are independently selected from the group consisting of hydrogen and an alkyl group. In an additional alternative of this embodiment, R³, R⁴, R⁷, and R⁸ are independently selected from the group consisting of hydrogen and methyl. In a further alternative of this embodiment, R³, R⁴, R⁷, and R⁸ are each methyl. In yet another alternative of this embodiment, R³ and R⁴ are each methyl; and R⁷ and R⁸ are each hydrogen. In still another alternative of this embodiment, R³ and R⁴ are each hydrogen; and R⁷ and R⁸ are each methyl.

In a further exemplary embodiment for compounds having Formula (V), the compound comprises Formula (Va):

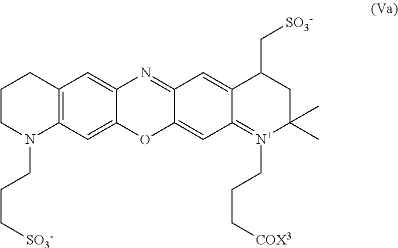

wherein:
X³ is selected from the group consisting of hydroxyl, a N-succinimidyl ester group, and a 2-(N-maleimidoethyl)amino group.

In another exemplary embodiment for compounds having Formula (V), the compound comprises Formula (Vb):

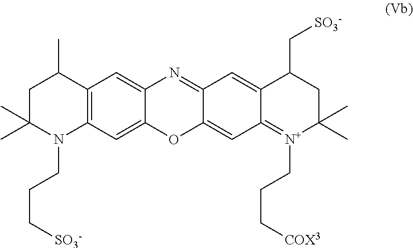

wherein:
X³ is selected from the group consisting of hydroxyl, a N-succinimidyl ester group, and a 2-(N-maleimidoethyl)amino group.

In a further exemplary embodiment for compounds having Formula (V), the compound comprises Formula (Vc):

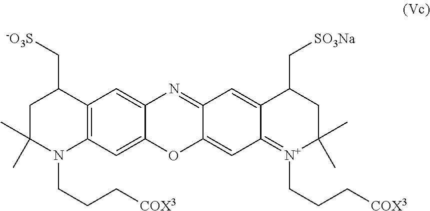

wherein:
X³ is selected from the group consisting of hydroxyl, a N-succinimidyl ester group, and a 2-(N-maleimidoethyl)amino group.

In a further embodiment, the compound comprises Formula (VI):

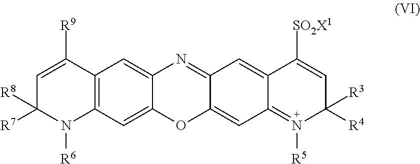

wherein:
R³, R⁴, R⁵, R⁶, R⁷, R⁸, R⁹, Rₓ, and L₁ are as described for compounds having Formula (I), and X¹ is selected from the group consisting of O⁻, ONa, OLi, OK, ONH₄, and ONR₄, wherein R is an alkyl group.

In one exemplary embodiment for compounds having Formula (VI), R³, R⁴, R⁷, and R⁸ are independently selected from the group consisting of hydrogen and an alkyl group. In an alternative of this embodiment, R³, R⁴, R⁷, and R⁸ are independently selected from the group consisting of hydrogen and methyl. In another alternative of this embodiment, R³, R⁴, R⁷, and R⁸ are each methyl. In still another alternative of this embodiment, R³ and R⁴ are each methyl; and R⁷ and R⁸ are each hydrogen. In an additional alternative of this embodiment, R³ and R⁴ are each hydrogen; and R⁷ and R⁸ are each methyl.

In an additional exemplary embodiment for compounds having Formula (VI), R⁵ and R⁶ are independently selected from the group consisting of {—}(CH₂)ₘCOX² and {—}(CH₂)ₘSO₃⁻; m is an integer from 1 to 5; X² is selected from the group consisting of hydroxyl, a N-succinimidyl ester group, and a 2-(N-maleimidoethyl)amino group; and one of R⁹ and R¹⁰ is hydrogen, and the other of R⁹ and R¹⁰ is selected from the group consisting of hydrogen, methyl, and {—}(CH₂)ₘSO₃⁻. In one alternative of this embodiment, R³, R⁴, R⁷, and R⁸ are independently selected from the group consisting of hydrogen and an alkyl group. In an additional alternative of this embodiment, R³, R⁴, R⁷, and R⁸ are independently selected from the group consisting of hydrogen and methyl. In a further alternative of this embodiment, R³, R⁴, R⁷, and R⁸ are each methyl. In yet another alternative of this embodiment, R³ and R⁴ are each methyl; and R⁷ and R⁸ are each hydrogen. In still another alternative of this embodiment, R³ and R⁴ are each hydrogen; and R⁷ and R⁸ are each methyl.

In a further exemplary embodiment for compounds having Formula (VI), the compound comprises Formula (VIa):

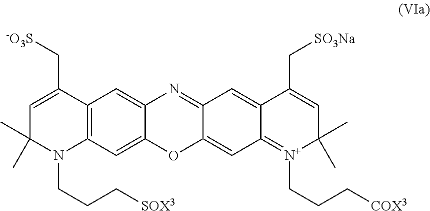

wherein:
X³ is selected from the group consisting of hydroxyl, a N-succinimidyl ester group, and a 2-(N-maleimidoethyl)amino group.

Suitable non-limiting examples of compounds having Formulas (I), (II), (III), (IV), (V), and (VI) are illustrated in Table 2 and in the examples.

TABLE 2

| Compound Number | Structure |
|---|---|
| Dye 2 carboxylic acid | 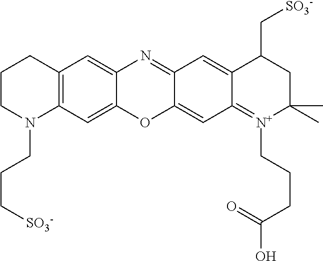 |
| Dye 2 succinimidyl ester | 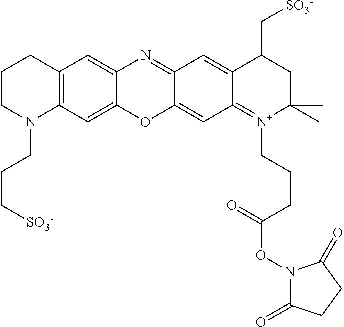 |
| Dye 2 maleimide | 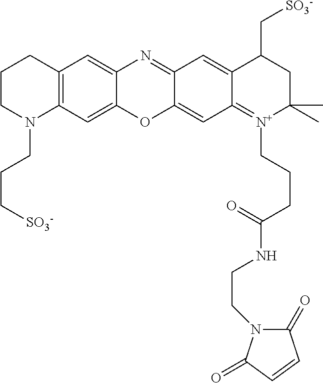 |
| Dye 3 carboxylic acid | 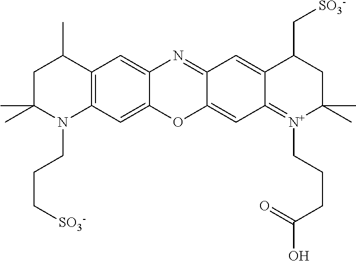 |
| Dye 3 succinimidyl ester | 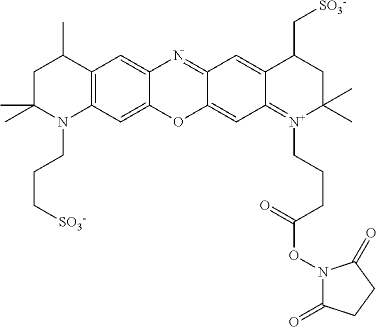 |
| Dye 3 maleimide | 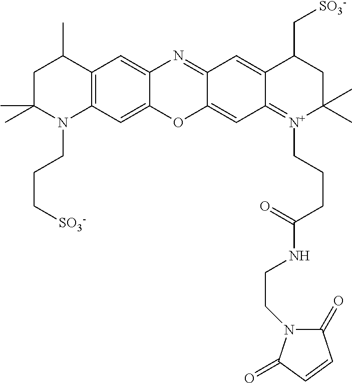 |
| Dye 6 | 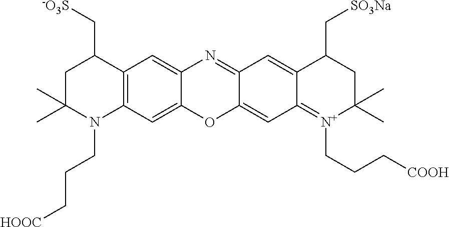 |
| Dye 7 | 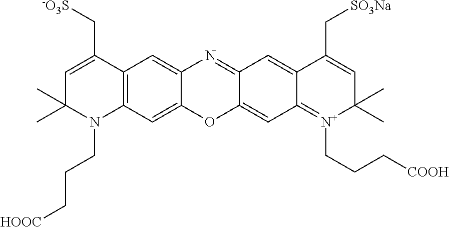 |
| Dye 10 | 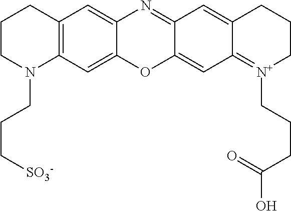 |
| Dye 14 carboxylic acid | 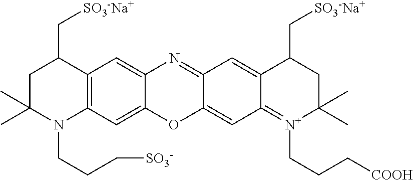 |
| Dye 14 succinimidyl ester | 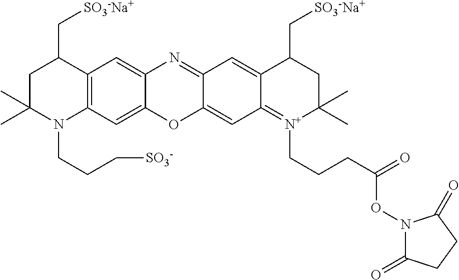 |

(II) Processes for Synthesizing Oxazine Dye Compounds

The dye compounds of the invention may be synthesized according to Reaction Scheme 1:

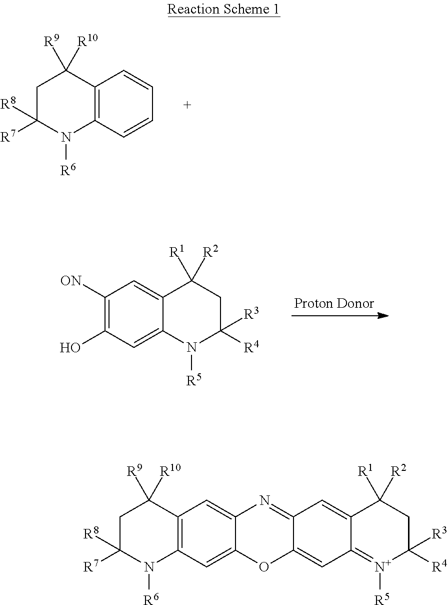

In a further embodiment, the dye compounds of the invention may be synthesized according to Reaction Scheme 2:

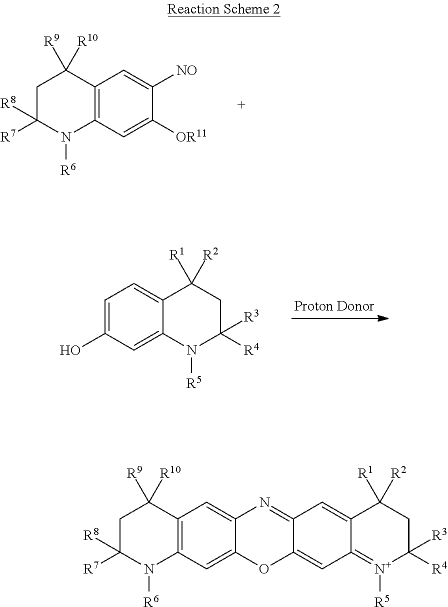

In an additional embodiment, the dye compounds of the invention may be synthesized according to Reaction Scheme 3:

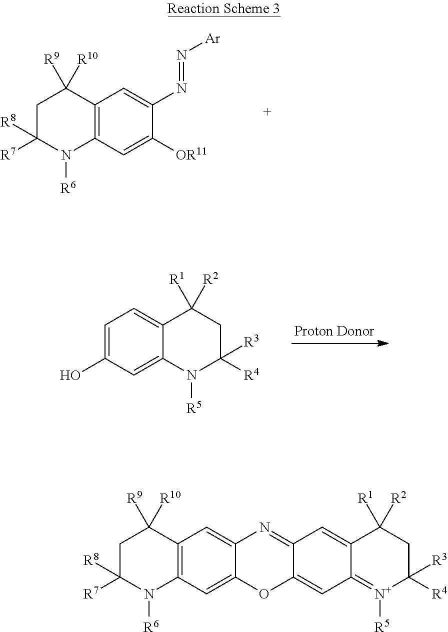

For each of Reaction Schemes 1, 2, and 3:
R¹, R², R³, R⁴, R⁵, R⁶, R⁷, R⁸, R⁹, R¹⁰ are as described in Section (I);
R¹¹ may be selected from alkyl and hydrogen;
Ar comprises C₆H₄Xₐ; and
Xₐ comprises an electron-withdrawing group. Suitable electron withdrawing groups include CN, Cl, and NO₂.

In general, the proton donor has a pKa less than about 6. Suitable proton donors include, but are not limited to, HOAc, HCO₂H, MeSO₃H, poly H₃PO₄, H₃PO₄, H₂SO₄, HCl, HBr, HI, HClO₄, CF₃SO₃H, and p-methyltoluenesulfonic acid.

The choice of solvent will depend upon the reactants. In general, the solvent may be a protic solvent, an aprotic solvent, or a combination thereof. Non-limiting examples of suitable protic solvents include methanol, ethanol, isopropanol, n-propanol, isobutanol, t-butanol, n-butanol, formic acid, acetic acid, and water. Non-limiting examples of aprotic solvents include ether solvents, acetone, acetonitrile, benzene, diethoxymethane, dimethylformamide (DMF), dimethyl sulfoxide (DMSO), N,N-dimethylpropionamide, 1,3-dimethyl-3,4,5,6-tetrahydro-2(1H)-pyrimidinone (DMPU), 1,3-dimethyl-2-imidazolidinone (DMI), 1,2-dimethoxyethane (DME), dimethoxymethane, dimethylacetamide (DMAC), N-methylpyrrolidinone (NMP), ethyl acetate, ethyl formate, ethyl methyl ketone, formamide, hexachloroacetone, hexamethylphosphoramide, methyl acetate, N-methylacetamide, N-methylformamide, methylene chloride, nitrobenzene, nitromethane, propionitrile, sulfolane, tetramethylurea, tetrahydrofuran (THF), toluene, trichloromethane.

In addition to the reactants delineated in Reaction Schemes 1, 2, and 3, any of a variety of reactants or reaction intermediates generally known in the art or specifically disclosed herein may be utilized to synthesize a dye compound of the invention. By way of non-limiting example, suitable compounds that may be used to synthesize the dye compounds are illustrated in Table 3. Methods for synthesizing each of these compounds are more fully described in the examples.

TABLE 3

| Com- | |
|---|---|
| pound | |
| Number | Structure |
| Inter- mediate 1 | 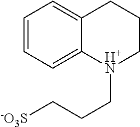 |
| Inter- mediate 2 | 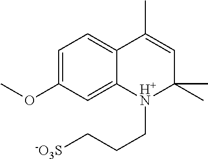 |
| Inter- mediate 3 | 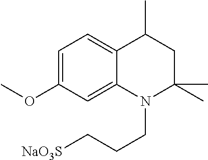 |
| Inter- mediate 4 | 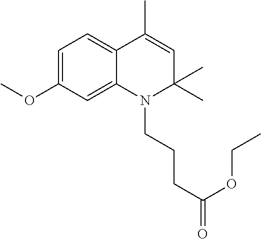 |
| Inter- mediate 5 | 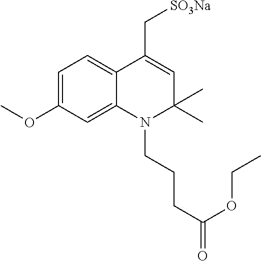 |
| Inter- mediate 6 | 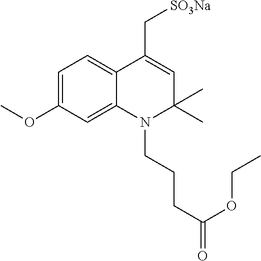 |
| Inter- mediate 7 | 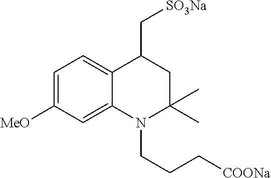 |
| Inter- mediate 8 | 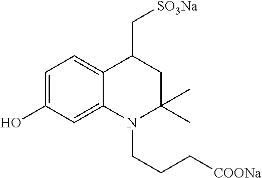 |
| Inter- mediate 9 | 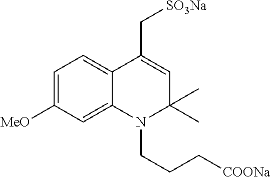 |
| Inter- mediate 10 | 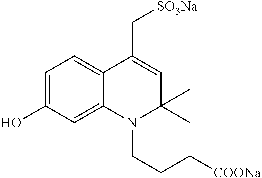 |
| Inter- mediate 11 | 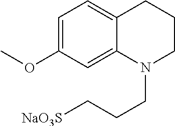 |
| Inter- mediate 12 | 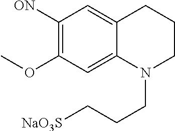 |
| Inter- mediate 13 | 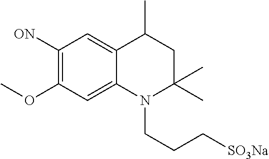 |
| Inter- mediate 14 | 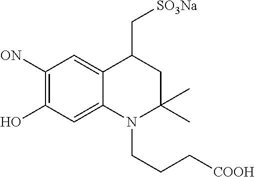 |
| Inter- mediate 15 | 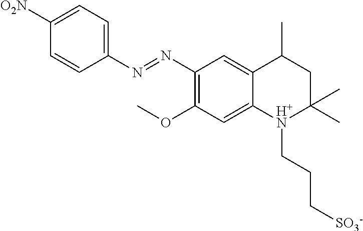 |
| Inter- mediate 16 | 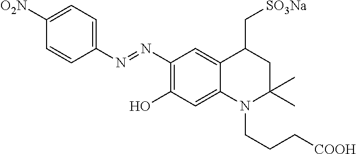 |
| Inter- mediate 17 | 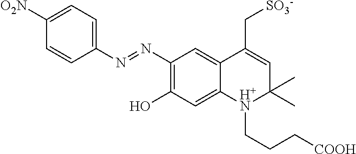 |
| Inter- mediate 18 | 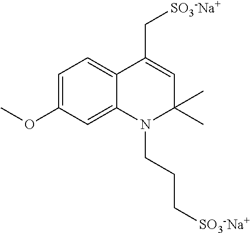 |
| Inter- mediate 19 | 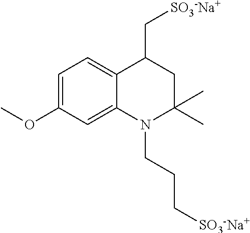 |
| Inter- mediate 20 | 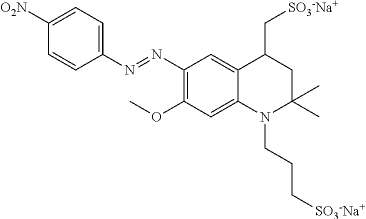 |

(III) Methods for Use of Oxazine Dye Compounds

A dye compound of the invention may be conjugated to at least one molecule (e.g., analyte or biomolecule) to form a probe, a sensor, or a detector. Non-limiting examples of suitable molecules include antigens, small molecules, steroids, vitamins, drugs, haptens, metabolites, analytes, toxins, environmental pollutants, amino acids, peptides, proteins, photosensitizers, nucleotides, oligonucleotides, nucleic acids, carbohydrates, lipids, ion-complexing moieties, synthetic polymers, dendrimer-based polymers, microparticles, and nanoparticles. In one exemplary embodiment, the molecule is a natural or synthetic amino acid or a natural or synthetic peptide or protein. Preferred peptides include, but are not limited to, protein kinase substrates, phosphatase substrates, protease substrates, neuropeptides, cytokines, and toxins. Preferred proteins include enzymes, antibodies, lectins, glycoproteins, histones, lipoproteins, biotin, avidin, streptavidin, protein A, protein G, casein, phycobiliproteins, fluorescent proteins (such as GFP, etc.), hormones, toxins, growth factors, and the like.

The point of attachment of the molecule to the dye compound can and will vary depending upon the embodiment. The type of attachment may be, for example, by covalent bonding, ionic bonding, dated bonding, hydrogen bonding, and other forms of molecular bonding. In preferred embodiments, the attachment may be by covalent bonding, as described in more detail in Section (I).

The dye compounds or conjugates thereof are useful in many biological applications. For example, the dye compounds may be used in imaging with techniques such as those based on fluorescence detection, including but not limited to fluorescence lifetime, anisotropy, photo-induced electron transfer, photobleaching recovery, and non-radioactive transfer. The dye compounds, as such, may be utilized in all fluorescent-based imaging, microscopy, and spectroscopy techniques including variations on such. In addition, they may also be used for photodynamic therapy and in multimodal imaging. Exemplary fluorescence detection techniques include those that involve detecting fluorescence generated within a system. Such techniques include, but are not limited to, fluorescence microscopy, confocal microscopy, multiphoton microscopy, fluorescence resonance energy transfer (FRET), total internal reflection fluorescence microscopy (TIRF), fluorescence activated cell sorting (FACS), fluorescent flow cytometry, fluorescence correlation spectroscopy (FCS), fluorescence in situ hybridization (FISH), multiphoton imaging, diffuse optical tomography, molecular imaging in cells and tissue, fluorescence imaging with one nanometer accuracy (FIONA), free radical initiated peptide sequencing (FRIPs), and second harmonic retinal imaging of membrane potential (SHRIMP), as well as other methods known in the art.

In certain embodiments, the dye compounds or conjugates thereof may be used to detect single molecules in cells. That is, the dye compound may be used to detect the presence or the location of the single molecule. Non-limiting examples of suitable molecules include proteins (such as, e.g., enzymes, receptors, cell surface proteins, cytoskeletal proteins, nuclear proteins, etc.), nucleic acids (such as mRNA, miRNA and other small RNAs), lipids (such as IP3, PIP3, and the like), carbohydrates, metabolites, or pathogens.

In other embodiments, the dye compounds or conjugates thereof may be used to monitor molecular interactions. Examples of molecular interactions include protein-protein interactions, enzyme-substrate interactions, receptor-ligand interactions, cell signaling events, protein-nucleic acid interactions, host-pathogen interactions, and so forth. The detection and imaging of such molecular interactions may be conducted in vitro or in vivo. As an example, the dye compounds may be used to detect the activity of an enzyme, such as the transfer of a phosphate group to a substrate by a kinase. Examples of other enzymes whose activity may be monitored include phosphatases, hydrolases, capsases, proteases, nucleases, polymerases, ligases, transferases, synthetases, lipases, and so forth.

In still other embodiments, the dye compounds or conjugates thereof may be used as markers or tags to track dynamic behavior in living cells. In this regard, fluorescence recovery after photobleaching (FRAP) may be employed in combination with the subject fluorescent dye compounds to selectively destroy fluorescent molecules within a region of interest with a high-intensity laser, followed by monitoring the recovery of new fluorescent molecules into the bleached area over a period of time with low-intensity laser light. Variants of FRAP include, but are not limited to, polarizing FRAP (pFRAP), fluorescence loss in photo-bleaching (FLIP), fluorescence localization after photobleaching (FLAP). The resulting information from FRAP and variants of FRAP can be used to determine kinetic properties, including the diffusion coefficient, mobile fraction, and transport rate of the fluorescently labeled molecules.

In additional embodiments, the dye compounds or conjugates thereof may be used to track assembly of cellular structures in vitro or in vivo. The cellular structure may be a cytoskeletal structure, such as those formed by actin, tubulin, or intermediate filaments. Tracking cytoskeletal assembly permits the tracking of cell movement, cell motility, protein trafficking, vesicular transport, membrane vesicle transport, mitotic spindle assembly, cytokinesis, and other such cytoskeletal-mediated process.

### DEFINITIONS

To facilitate understanding of the invention, several terms are defined below.

The term “acyl,” as used herein alone or as part of another group, denotes the moiety formed by removal of the hydroxy group from the group COOH of an organic carboxylic acid, e.g., RC(O), wherein R is R¹, R¹O—, R¹R²N—, or R¹S—, R¹ is hydrocarbyl, heterosubstituted hydrocarbyl, or heterocyclo, and R² is hydrogen, hydrocarbyl or substituted hydrocarbyl.

The term “alkyl” as used herein describes groups which are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “alkenyl” as used herein describes groups which are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The term “alkynyl” as used herein describes groups which are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “aromatic” as used herein alone or as part of another group denotes optionally substituted homo- or heterocyclic aromatic groups. These aromatic groups are preferably monocyclic, bicyclic, or tricyclic groups containing from 6 to 14 atoms in the ring portion. The term “aromatic” encompasses the “aryl” and “heteroaryl” groups defined below.

The term “aryl” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The terms “halogen” or “halo” as used herein alone or as part of another group refer to chlorine, bromine, fluorine, and iodine.

The term “heteroatom” shall mean atoms other than carbon and hydrogen.

The terms “heterocyclo” or “heterocyclic” as used herein alone or as part of another group denote optionally substituted, fully saturated or unsaturated, monocyclic or bicyclic, aromatic or non-aromatic groups having at least one heteroatom in at least one ring, and preferably 5 or 6 atoms in each ring. The heterocyclo group preferably has 1 or 2 oxygen atoms and/or 1 to 4 nitrogen atoms in the ring, and is bonded to the remainder of the molecule through a carbon or heteroatom. Exemplary heterocyclo groups include heteroaromatics as described below. Exemplary substituents include one or more of the following groups: hydrocarbyl, substituted hydrocarbyl, hydroxy, protected hydroxy, acyl, acyloxy, alkoxy, alkenoxy, alkynoxy, aryloxy, halogen, amido, amino, cyano, ketals, acetals, esters and ethers.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, aryloxy, hydroxy, protected hydroxy, acyl, acyloxy, nitro, amino, amido, nitro, cyano, ketals, acetals, esters and ethers.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above compounds and processes without departing from the scope of the invention, it is intended that all matter contained in the above description and in the examples given below, shall be interpreted as illustrative and not in a limiting sense.

### EXAMPLES

The following examples illustrate various embodiments of the present invention.

### Example 1

### Synthesis of Dye 2

The total synthesis of Dye 2 is depicted in the following reaction scheme:

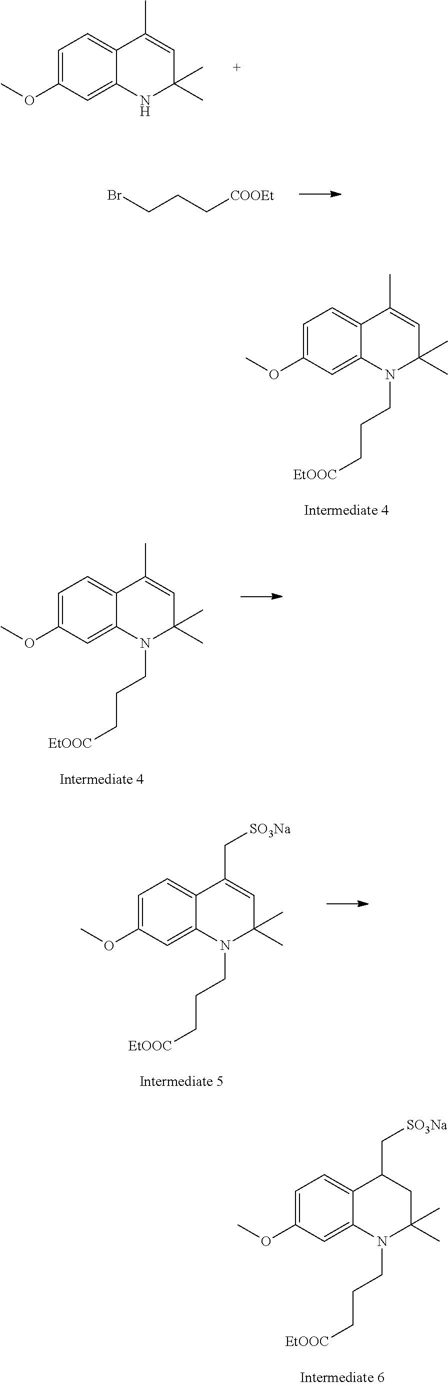

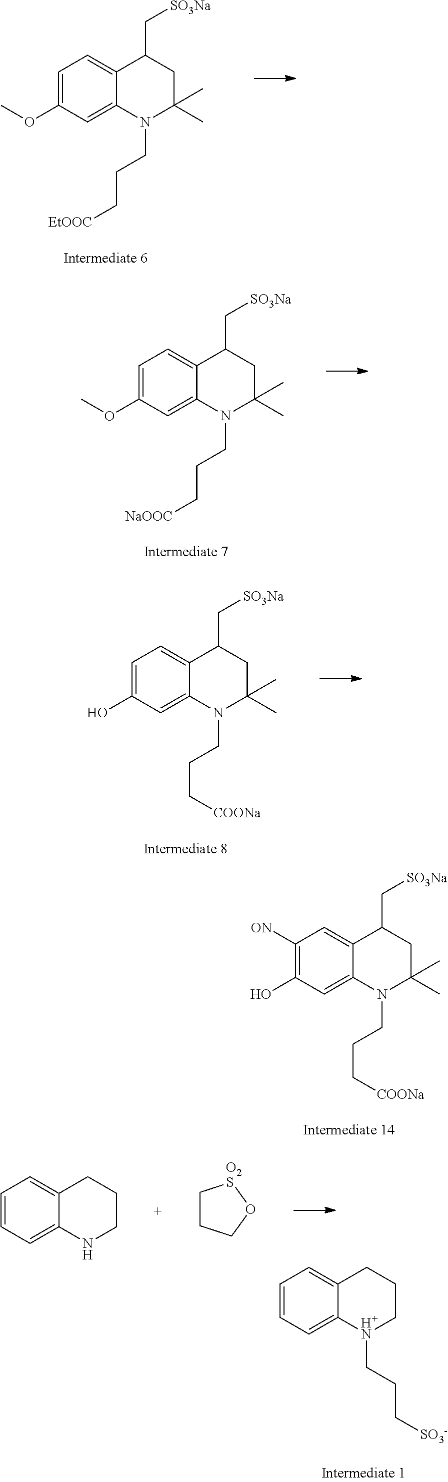

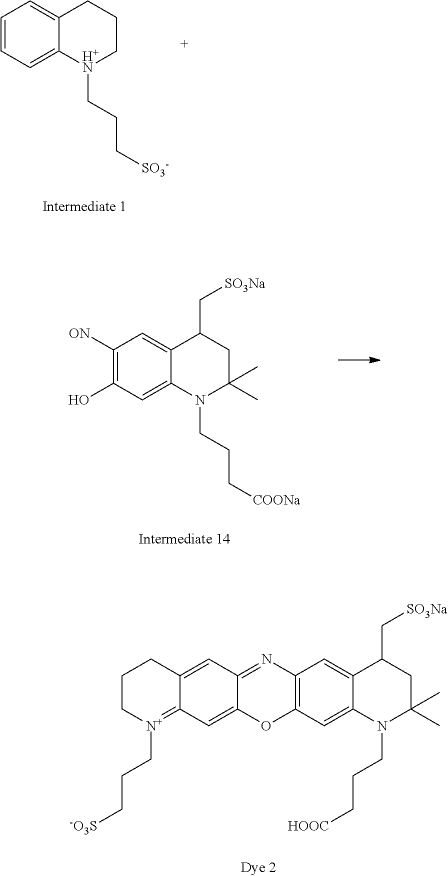

Individual steps of the process are presented in more detail below in Examples 4, 6-10, 17, and 19.

### Example 2

### Synthesis of Dye 3

The following reaction scheme depicts the total synthesis of Dye 3:

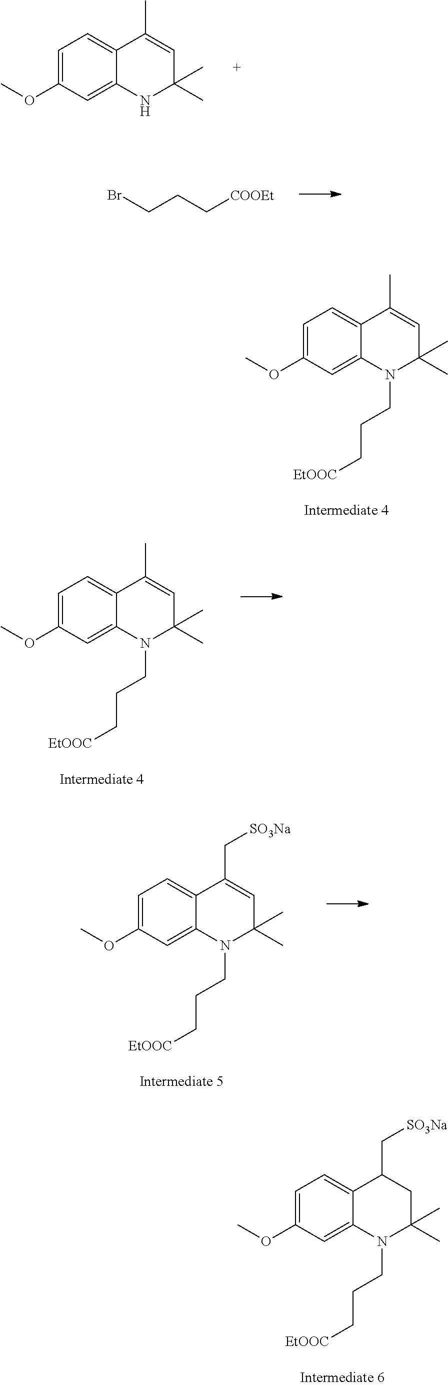

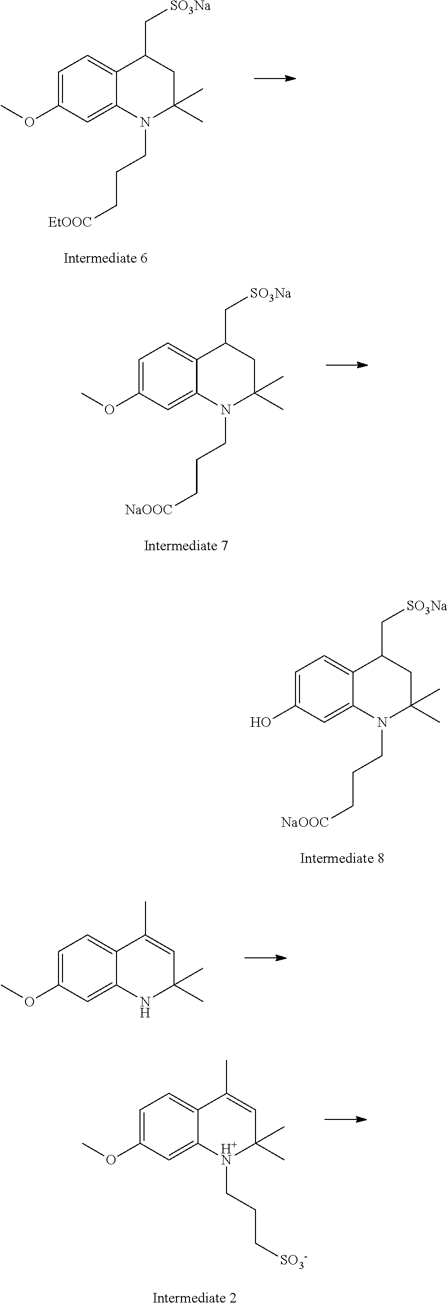

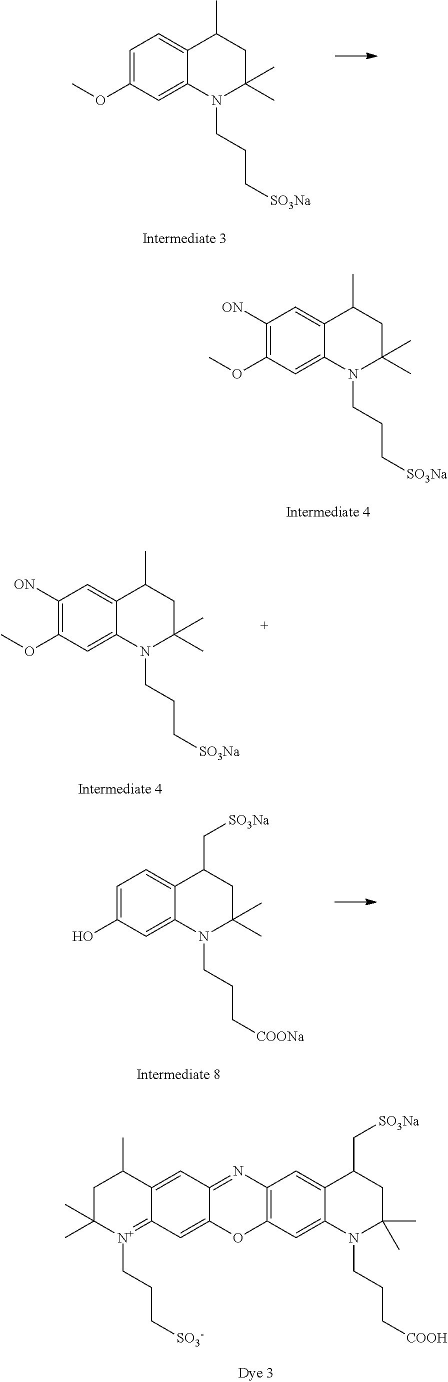

Individual steps of the reaction scheme are detailed below in Examples 4-10, 15, and 20.

### Example 3

### Synthesis of Intermediate 1

The following reaction scheme depicts the synthesis of intermediate 1(3-(3,4-dihydroquinolinium-1(2H)-yl)propane-1-sulfonate):

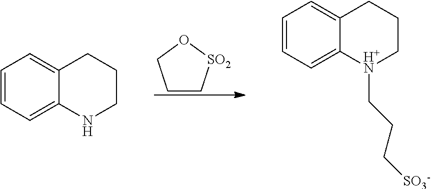

A round-bottom, one-necked 500 mL flask was set up with a magnetic stir bar, an oil-heating bath, and a thermometer. 1,2,3,4-Tetrahydroquinoline (13.3 g, 0.100 mol) was added to the flask and stirring was started. Next 1,3-propane sultone (14.6 g, 0.120 mol) was added drop-wise over the course of 1 minute. After about 5 minutes, the temperature of the reaction mixture rose to about 140° C. After about 10 minutes, the reaction mixture became very viscous and the stirring was discontinued. The flask was immersed in the oil bath (T=160° C.) and incubated for another 30 minutes without stirring. The flask was cooled to room temperature, and the yellow solid residue was carefully broken into large pieces using a spatula. Methanol (100 mL) was added to the flask and a reflux condenser was attached to the flask. The mixture was stirred under reflux for 30 minutes. The mixture was cooled to room temperature. White crystals of the product (about 98% purity based on ¹H NMR) were filtered on Buchner funnel, washed with methanol (20 mL), and dried. The yield was 18.0 g (71% of theoretical yield).

Analysis. ¹H NMR (D₂O, 300 MHz) δ 2.15 (m, 4H, CH₂, CH₂), 2.91 (m, 4H, CH₂, CH₂), 3.61 (m, 4H, CH₂, CH₂), 7.30 (m, 4H, aromatic).

### Example 4

### Synthesis of Intermediate 2

Intermediate 2 (3-(7-methoxy-2,2,4-trimethylquinolinium-1(2H)-yl)propane-1-sulfonate) was synthesized according to the following reaction scheme:

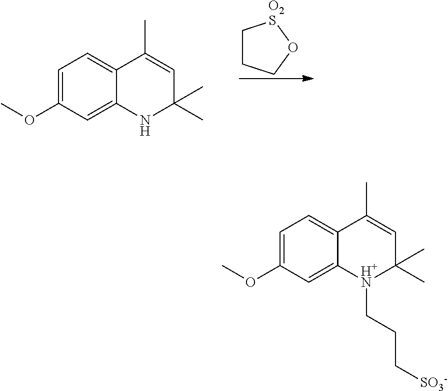

7-Methoxy-2,2,4-trimethyl-1,2-dihydro-quinoline (20.3 g, 0.100 mol) and 1,3-propane sultone (14.6 g, 0.120 mol) were placed in a 500 mL round-bottom one-necked flask equipped with a magnetic stir bar. The flask was loosely closed with a stopper and the mixture was heated at 145° C. (i.e., in an oil bath) for 3 hour, at which time the reaction mixture was very viscous and the stirring was stopped. The mixture was cooled to room temperature, and 100 mL of methanol was added to the flask. The flask was heated in an oil bath (T=100° C.) until a clear solution was formed. The mixture was cooled to room temperature and a white precipitate was formed. The white precipitate was filtered, washed with methanol, and dried. The yield was 22.0 g. (67.6%).

Analysis. ¹H NMR (MeOD-D₂O, 300 MHz) δ 1.54 (s, 6H, 2*CH₃), 1.96 (m, 2H), 2.14 (s, 3H, CH₃), 2.85 (t, 2H, ³J_{H-H}=6.9 Hz), 3.59 (t, 2H, ³J_{H-H}=7.2 Hz), 3.91 (s, 3H, CH₃O), 5.62 (s, 1H), 7.0-7.1 (m, 2H), 7.54 (s, 1H). ¹³C NMR (MeOD-d4, 75 MHz) δ 17.8, 22.4, 24.1, 50.1, 51.9, 56.7, 64.7, 111.8, 116.8, 121.9, 125.7, 128.2, 128.3, 132.7, 134.9, 161.5.

### Example 5

### Synthesis of Intermediate 3

The synthesis of intermediate 3 (sodium 3-(7-methoxy-2,2,4-trimethyl-3,4-dihydroquinolin-1(2H)-yl)propane-1-sulfonate) is depicted in the reaction scheme presented below:

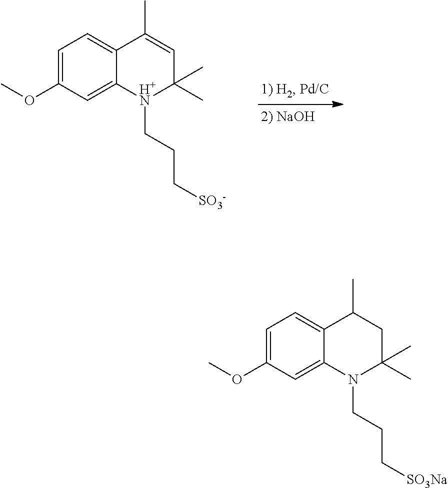

Intermediate 2 (3-(7-methoxy-2,2,4-trimethylquinolinium-1(2H)-yl)propane-1-sulfonate) (7.0 g, 0.022 mol), methanol (250 mL, 6.2 mol), and Pd on carbon (10%, 1.00 g) were added to a 1 L high pressure, heavy wall Parr hydrogenation bottle. The bottle was attached to a high-pressure Parr hydrogenation apparatus and the system was evacuated using a water aspirator and then filled with hydrogen gas. The evacuation/filling procedure was repeated three more times. The system then was filled with hydrogen at 50 psi pressure and the shaker was activated. The bottle was shaken at room temperature for 4 hours (at which time hydrogen consumption had stopped). The remaining pressure was carefully released and the catalyst was removed by vacuum filtration through a Celite pad. The solvent was removed under vacuum and the resultant residue was treated with solution of sodium hydroxide (0.860 g, 0.0215 mol) in water (25 mL). The mixture was heated until a clear solution formed. Upon cooling the product crystallized. The crystals were washed with cold water, air dried on a Buchner funnel, and then dried under high vacuum overnight. The yield was 3.8 g (50% of theoretical yield).

Analysis. ¹H NMR (D₂O, 300 MHz) δ 1.15 (s, 3H, CH₃), 1.21 (d, 3H, ³J_{H-H}=6.9 Hz), 1.42 (s, 3H, CH₃), 1.7 (m, 1H), 1.9-2.1 (m, 3H), 2.6-3.0 (m, 3H), 3.3 (m, 1H), 3.69 (s, 3H), 6.71 (d, 1H, ⁴J_{H-H}=2.4 Hz), 6.95 (dd, 1H, ³J_{H-H}=8.7 Hz, ⁴J_{H-H}=2.4 Hz), 7.32 (d, 1H, ³J_{H-H}=8.7 Hz).

### Example 6

### Synthesis of Intermediate 4

Intermediate 4 (4-(7-methoxy-2,2,4-trimethyl-2H-quinolin-1-yl)-butyric acid ethyl ester) was formed as depicted in the following reaction scheme:

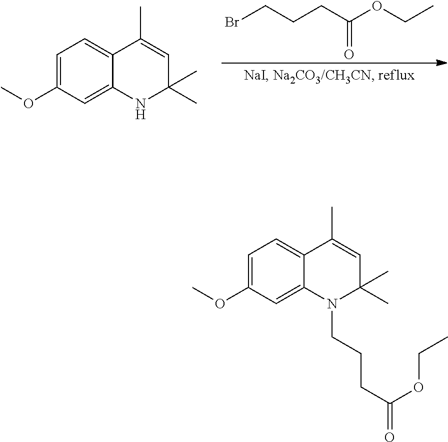

A 500 mL round-bottom flask was set up with a magnetic stir bar, a reflux condenser, and a nitrogen line. The flask was charged with 7-methoxy-2,2,4-trimethyl-1,2-dihydro-quinoline (43 g, 0.21 mol), ethyl 4-bromobutyrate (44 g, 0.22 mol), sodium iodide (11 g, 0.073 mol), acetonitrile (150 mL, 2.9 mol), and sodium carbonate (26 g, 0.24 mol). The flask was heated at reflux with stirring for 72 hours. The progress of the reaction was monitored by thin layer chromatography (silica-gel plates, CH₂Cl₂ as eluent, R_{f} starting compound=0.29; R_{f} product=0.20). After 20 hours the conversion was about 50%. Additional ethyl 4-bromobutyrate (44 g) and sodium carbonate (26 g) were added to the reaction mixture and reflux was continued for additional 48 hours. TLC analysis showed about 85% conversion.

The reaction mixture was filtered and the acetonitrile was removed with a rotary evaporator. The unreacted starting compound was removed from the product by vacuum distillation using an oil pump. The recovery of 7-methoxy-2,2,4-trimethyl-1,2-dihydro-quinoline was 7 g (16%, b.p. 120-125° C., 0.7 Torr). The viscous yellow residue in the distillation flask was the pure product. The yield was 53.0 g oil (79% of theoretical yield).

Analysis. ¹H NMR (CDCl₃, 300 MHz) δ 1.25-1.30 (m, 9H, 2*CH₃, CH₃), 1.92-1.94 (m, 5H, CH₃, CH₂), 2.38 (t, 2H, ³J_{H-H}=6.9 Hz, CH₂CO₂CH₂CH₃), 3.23 (distorted t, 2H, CH₂N), 3.79 (s, 3H, CH₃O), 4.16 (q, ³J_{H-H}=7.2 Hz), 5.11 (s, 1H, CH), 6.12-6.15 (m, 2H), 6.97 (d, 1H, ³J_{H-H}=8.1 Hz). ¹³C NMR (CDCl₃, 75 MHz) δ 14.2, 18.6, 23.4, 28.3, 31.7, 43.4, 55.1, 56.8, 60.4, 98.0, 99.4, 116.9, 124.4, 127.1, 127.5, 145.2, 160.6, 173.2.

### Example 7

### Synthesis of Intermediate 5

The following reaction scheme depicts the preparation of intermediate 5 (sodium[1-(3-ethoxycarbonyl-propyl)-7-methoxy-2,2-dimethyl-1,2-dihydro-quinolin-4-yl]-methanesulfonate):

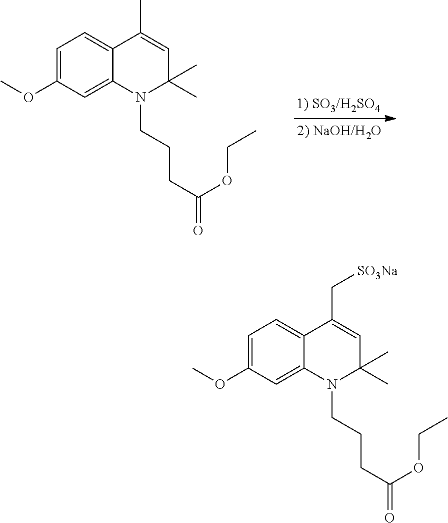

A 500 mL round-bottom, three-necked flask was set up with a nitrogen line, a mechanical stirrer, and a thermometer. The flask was immersed in a salt-ice cooling bath and intermediate 4 (4-(7-methoxy-2,2,4-trimethyl-2H-quinolin-1-yl)-butyric acid ethyl ester) (30 g, 0.09 mol) was placed in the flask and the stirrer was started. Sulfuric acid (30 mL, 0.6 mol) was added slowly to the flask over the course of about 5 minutes. The reaction mixture was then stirred for additional 30 minutes with cooling. When the temperature inside the flask was about −10° C., fuming sulfuric acid (17 mL, 0.18 mol, 30% of free SO₃) was added quickly to the flask. The stirring was continued and the flask was allowed to slowly warm up to room temperature while in cooling-bath. The reaction mixture was stirred for another 48 hours at room temperature.

The reaction mixture was poured into a 2 L beaker containing 500 g of ice and the beaker was immersed in a salt-ice cooling bath. The acid was neutralized with 10% solution of sodium hydroxide in water until the pH of the reaction mixture was about 7. The temperature of the reaction mixture was kept below 10° C. during the neutralization process, at the end of which the product crystallized. The crystals were filtered using a Buchner funnel, washed with 50 mL of ice-cold water, and air-dried. The crystals were placed in 2 L beaker and 1 L of 100% ethanol was added. The mixture was stirred with heating until the ethanol started to boil and some of the solid material dissolved. The mixture was filtered while still hot using a Buchner funnel. The crystals (Na₂SO₄ hydrate) were washed with 100 mL of hot ethanol and discarded. The solution was evaporated using a rotary evaporator and the resultant white solid was dried under high vacuum overnight. The yield was 26 g (66% of theoretical yield).

Analysis. ¹H NMR (dmso-d6, 300 MHz) δ 1.16-1.25 (m, 9H, 2*CH₃, CH₃), 1.7-1.9 (m, 2H, CH₃, CH₂), 2.41 (t, 2H, ³J_{H-H}=6.6 Hz, CH₂CO₂CH₂CH₃), 3.17 (distorted t, 2H, CH₂N), 3.45 (s, 2H, CH₂SO₃), 3.70 (s, 3H, CH₃O), 4.08 (q, ³J_{H-H}=7.2 Hz, CH₂CH₃), 5.31 (s, 1H, CH), 6.05-6.15 (m, 2H), 7.17 (d, 1H, ³J_{H-H}=8.1 Hz). ¹³C NMR (dmso-d6, 75 MHz) δ 14.0, 23.0, 27.7, 30.7, 42.7, 53.8, 54.6, 56.3, 59.7, 97.0, 99.4, 115.4, 125.2, 125.8, 129.9, 144.8, 159.7, 172.7.

### Example 8

### Synthesis of Intermediate 6

Intermediate 6 (sodium[1-(3-ethoxycarbonyl-propyl)-7-methoxy-2,2-dimethyl-1,2-dihydro-quinolin-4-yl]-methanesulfonate) was synthesized according to the following reaction scheme:

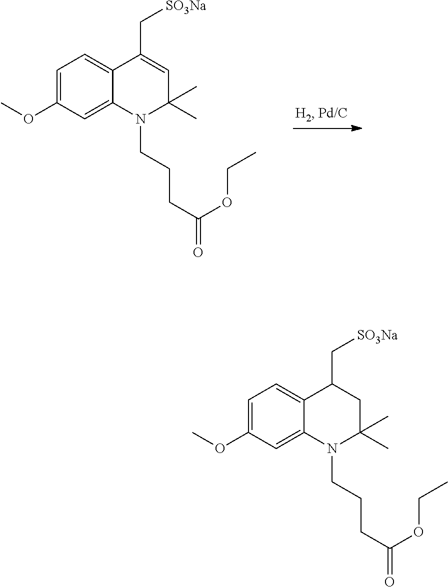

Intermediate 5 (sodium[1-(3-ethoxycarbonyl-propyl)-7-methoxy-2,2-dimethyl-1,2-dihydro-quinolin-4-yl]-methanesulfonate (22 g, 0.052 mol), methanol (250 mL, 6.2), and Pd on carbon (10%, 1.00 g) were added to a 1 L high pressure, heavy wall Parr hydrogenation bottle. The bottle was attached to a high-pressure Parr hydrogenation apparatus and the system was evacuated using a water aspirator and then filled with hydrogen gas. The evacuation/filling procedure was repeated three more times. Then the system was filled with hydrogen at 50 psi pressure and the shaker was activated. The bottle was shaken at room temperature for 4 hours until hydrogen consumption stopped. The remaining pressure was carefully released and the catalyst was removed by vacuum filtration through a Celite pad. The solvent was removed under vacuum to give the final product as a white solid. The yield was 20 g (90% of theoretical yield).

Analysis. ¹H NMR (D₂O-d6, 300 MHz) δ 0.97 (s, 3H), 1.0-1.25 (m, 6H, CH₃, CH₃), 1.40 (t, 1H, ³J_{H-H}=12.6 Hz), 1.5-1.7 (m, 2H), 2.0-2.2 (m, 3H), 2.7-2.8 (m, 2H), 3.0-3.2 (m, 2H), 3.4-3.6 (m, 4H), 4.0 (q, 2H, ³J_{H-H}=6.6 Hz), 6.02 (s, 1H), 6.14 (d, 1H, ³J_{H-H}=8.7 Hz), 6.96 (d, 1H, ³J_{H-H}=8.7 Hz).

### Example 9

### Synthesis of Intermediate 7

The reaction scheme shown below depicts the synthesis of intermediate 7 (disodium 4-(7-methoxy-2,2-dimethyl-4-(sulfonatomethyl)-3,4-dihydroquinolin-1(2H)-yl)butanoate):

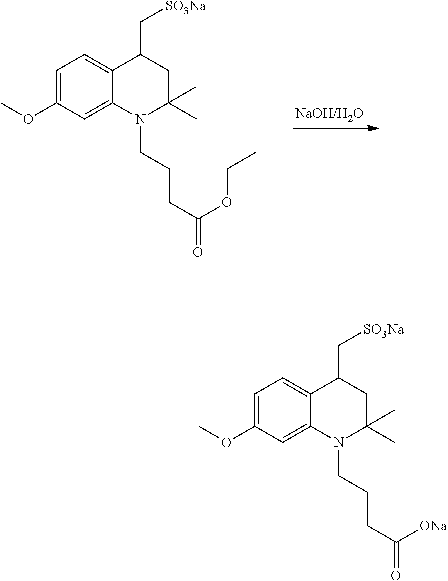

A 500 mL round-bottomed, one-necked flask was set up with a magnetic stir bar and a reflux condenser. The flask was charged with intermediate 6 (sodium[1-(3-ethoxycarbonyl-propyl)-7-methoxy-2,2-dimethyl-1,2-dihydro-quinolin-4-yl]-methanesulfonate) (20 g, 0.05 mol), sodium hydroxide (2.09 g, 0.0522 mol), and 100 mL water. The mixture was stirred with heating (50° C.) for 1 hour and then cooled to room temperature. The solvent was removed under vacuum and the solid was stirred with 100 mL of MeOH at reflux for 30 min. The solid was filtered, washed with methanol, air dried, and dried under high vacuum overnight. The yield of the final product (white solid) was 20 g (100% of theoretical yield).

Analysis. ¹H NMR (D₂O, 300 MHz) δ 1.08 (s, 3H, CH₃), 1.28 (s, 3H, CH₃), 1.60 (t, 1H, ³J_{H-H}=12.6 Hz), 1.8-2.0 (m, 2H), 2.1-2.4 (m, 3H), 2.8-3.1 (m, 2H), 3.2-3.4 (m, 2H), 3.55 (dd, ²J_{H-H}=14.4 Hz, ³J_{H-H}=3.0 Hz), 5.98 (d, 1H, ⁴

### Example 10

### Synthesis of Intermediate 8

Intermediate 8 (disodium 4-[7-hydroxy-2,2-dimethyl-4-(sulfonatomethyl)-3,4-dihydroquinolin-1(2H)-yl]butanoate) was prepared according to the reaction scheme presented below:

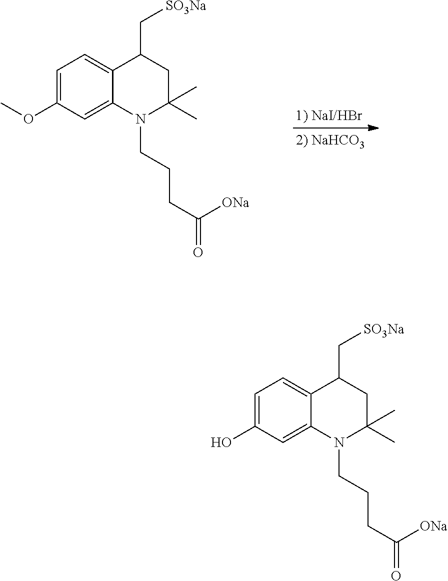

A 250 mL round-bottomed, one-necked flask was set up with a magnetic stir bar, a reflux condenser, and a nitrogen line. The flask was charged with intermediate 7 (disodium 4-(7-methoxy-2,2-dimethyl-4-(sulfonatomethyl)-3,4-dihydroquinolin-1(2H)-yl)butanoate) (12 g, 0.029 mol), sodium iodide (10 g, 0.07 mol), and a solution of 9.0 M hydrogen bromide in water (70 mL). The flask was flushed with nitrogen and then was heated at 105° C. (oil bath temperature) with stirring for 18 hours. The reaction mixture was cooled to room temperature and the excess hydrobromic acid was removed under vacuum. To ensure complete removal of the hydrobromic acid, 100 mL of water was added to the residue and the solvent was removed under vacuum. The yellow solid was dissolved in 50 mL of water and the solution was neutralized by addition of solid sodium bicarbonate. The solvent was removed by vacuum and the solid was refluxed with stirring with 500 mL of acetone overnight under nitrogen to remove any impurities of sodium iodide and sodium bromide. The mixture was cooled to room temperature and filtered. The solid was dried using a vacuum pump. The yield was 9.3 g (80% of theoretical yield).

Analysis. ¹H NMR (D₂O, 300 MHz) δ 1.30 (s, 3H, CH₃), 1.60 (s, 3H, CH₃), 1.8-2.2 (m, 2H, CH₂), 2.3-2.6 (m, 4H), 3.3-3.4 (m, 2H), 3.5-3.7 (m, 2H), 3.75-3.85 (m, 1H), 6.78 (d, 1H, ⁴J_{H-H}=2.1 Hz), 7.07 (dd, ³J_{H-H}=8.4 Hz, ⁴J_{H-H}=2.1 Hz), 7.47 (d, 1H, ³J_{H-H}=8.4 Hz). ¹³C NMR (MeOD-d4, 75 MHz) δ 18.4, 24.5, 27.3, 29.7, 31.0, 36.3, 45.6, 46.3, 55.1, 58.3, 58.5, 100.4, 103.7, 117.5, 127.9, 147.7, 157.5, 182.5.

### Example 11

### Synthesis of Intermediate 9

The synthesis of intermediate 9 (disodium 4-[7-methoxy-2,2-dimethyl-4-(sulfonatomethyl)quinolin-1(2H)-yl]butanoate) is depicted in the following reaction scheme:

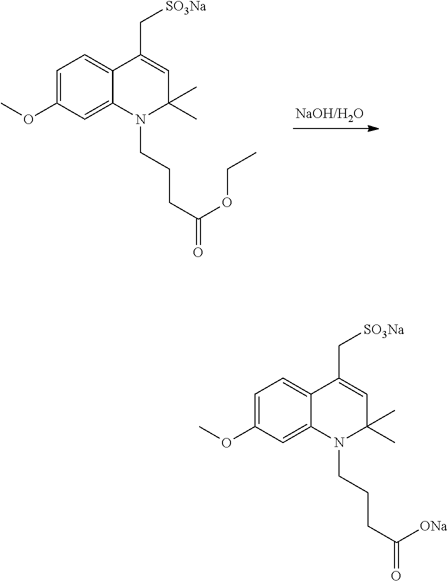

Intermediate 5 (sodium[1-(3-ethoxycarbonyl-propyl)-7-methoxy-2,2-dimethyl-1,2-dihydro-quinolin-4-yl]-methanesulfonate) (20 g, 0.05 mol) was added to a conical 500 mL flask equipped with a stir bar. Sodium hydroxide (2.3 g, 0.057 mol, 20% excess) was added to the flask. Water (100 mL) was added to the flask and the reaction mixture was stirred at room temperature for 8 hours. The water was removed using a rotary evaporator, and 200 mL of methanol was added to the white solid. The mixture was heated with stirring under reflux for 20 minutes and then cooled to room temperature. The solid was filtered, washed with methanol, and dried (using a vacuum pump overnight). The yield of the white solid final product was 16.0 g (80% of theoretical yield).

Analysis. ¹H NMR (D₂O, 300 MHz) δ 1.34 (s, 6H, 2*CH₃), 1.84 (p, 2H, ³J_{H-H}=6.6 Hz, CH₂CH₂CH₂CO₂), 2.29 (t, 2H, ³J_{H-H}=7.2 Hz, CH₂CO₂), 3.26 (distorted t, 2H, CH₂N), 3.85 (s, 3H, CH₃O), 3.97 (s, 2H, CH₂SO₃), 5.62 (s, 1H, CH), 6.31 (d, 1H, ⁴J_{H-H}=2.1 Hz), 6.38 (dd, ³J_{H-H}=8.4 Hz, ⁴J_{H-H}=2.1 Hz), 7.30 (d, 1H, ³J_{H-H}=8.4 Hz).

### Example 12

### Synthesis of Intermediate 10

The following reaction scheme depicts the preparation of intermediate 10 (disodium 4-(7-hydroxy-2,2-dimethyl-4-(sulfonatomethyl)quinolin-1(2H)-yl)butanoate):

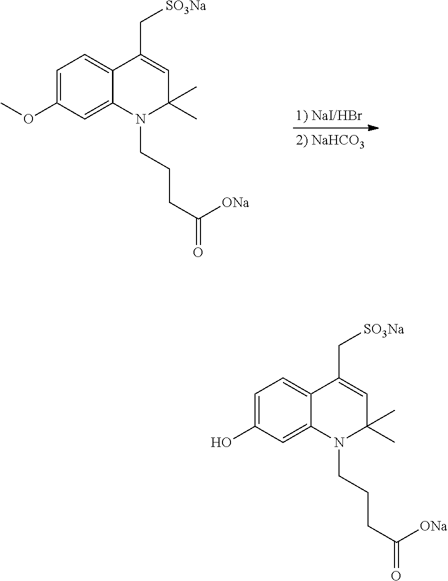

A 250 mL round-bottomed, one-necked flask was set up with a magnetic stir bar, a reflux condenser, and a nitrogen line. Intermediate 9 (disodium 4-[7-methoxy-2,2-dimethyl-4-(sulfonatomethyl)quinolin-1(2H)-yl]butanoate) (17.3 g, 0.0418 mol), sodium iodide (18.8 g, 0.126 mo), and hydrobromic acid (70 mL, 1 mol) were added to the flask. The flask was flushed with nitrogen and then heated in an oil bath (T=105° C.) with stirring for 18 hours. The reaction mixture was cooled to room temperature and the excess of hydrobromic acid was removed under vacuum. To ensure complete removal of hydrobromic acid, 100 mL of water was added to the residue and the solvent was removed under vacuum. The yellow solid was dissolved in 50 mL of water and the solution was neutralized by addition of solid sodium bicarbonate. The solvent was removed using a rotary evaporator. The solid was refluxed with stirring with 500 mL of acetone overnight under nitrogen to dissolve any impurities of sodium iodide and sodium bromide. The mixture was cooled to room temperature and filtered. The solid was dried using a vacuum pump. The yield of the gray solid product was 12.0 g (72% of theoretical yield).

Analysis. ¹H NMR (D₂O, 300 MHz) δ 1.34 (s, 6H, 2*CH₃), 1.84 (p, 2H, ³J_{H-H}=6.6 Hz, CH₂CH₂CH₂CO₂), 2.29 (t, 2H, ³J_{H-H}=7.2 Hz, CH₂CO₂), 3.26 (distorted t, 2H, CH₂N), 3.85 (s, 3H, CH₃O), 3.97 (s, 2H, CH₂SO₃), 5.62 (s, 1H, CH), 6.31 (d, 1H, ⁴J_{H-H}=2.1 Hz), 6.38 (dd, ³J_{H-H}=8.4 Hz, ⁴J_{H-H}=2.1 Hz), 7.30 (d, 1H, ³J_{H-H}=8.4 Hz).

### Example 13

### Synthesis of Intermediate 11

Intermediate 11 (sodium 3-(7-methoxy-3,4-dihydro-2H-quinolin-1-yl)-propane-1-sulfonate) was synthesized according to the following reaction scheme:

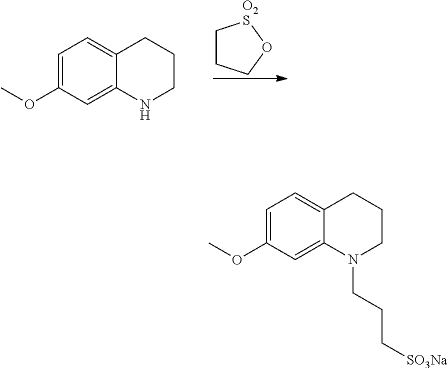

7-Methoxy-1,2,3,4-tetrahydro-quinoline (2.7 g, 0.016 mol) was added to a 100 mL round-bottomed, one-necked flask equipped with a magnetic stir bar. Then 1,3-propane sultone (3.8 g, 0.031 mol) was added to the flask, and the mixture was heated at 120° C. for 30 minutes with stirring under nitrogen. The flask was cooled, 25 ml of water was added and a clear solution formed. Solid sodium bicarbonate (2.0 g) was added slowly to the mixture to avoid excessive foaming. The mixture was stirred and heated to about 60° C. until the solids were dissolved. Then the mixture was cooled in ice-water for 1 hour, wherein the product separated as silvery glistening plates. The product was filtered, washed with ice water (about 5 mL), and dried. The yield was 3.23 g (64% of theoretical yield).

Analysis. ¹H NMR (dmso-d6, 300 MHz) δ 1.7-1.9 (m, 4H, CH₂, CH₂), 2.4-2.6 (m, 4H, CH₂, CH₂), 3.19 (br s, 2H), 3.30 (t, ³J_{H-H}=6.9 Hz, 2H), 3.66 (s, 3H, CH₃O), 6.02 (d, ³J_{H-H}=8.1 Hz, 1H), 6.17 (s, 1H), 6.73 (d, ³J_{H-H}=8.1 Hz, 1H).

### Example 14

### Synthesis of Intermediate 12

The reaction scheme shown below depicts the synthesis of intermediate 12 (sodium 3-(7-methoxy-6-nitroso-3,4-dihydroquinolin-1(2H)-yl)propane-1-sulfonate):

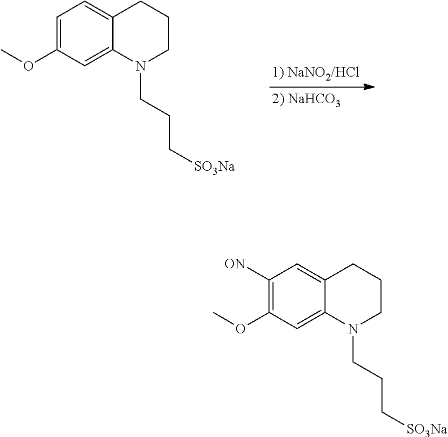

Intermediate 11 (sodium 3-(7-methoxy-3,4-dihydro-2H-quinolin-1-yl)-propane-1-sulfonate) (2.00 g, 0.00651 mol) was placed in a 250 mL round-bottomed, one-necked flask equipped with a magnetic stir bar and an addition funnel. A 12 M solution of hydrogen chloride in water (20 mL) was added. The mixture was stirred and the flask was immersed in an ice-water cooling bath. A solution of sodium nitrite (0.449 g, 0.00651 mol) was prepared in 10 mL of water. The sodium nitrite solution was placed in the addition funnel that was equipped with pressure equalizing arm. The solution of sodium nitrate was slowly added to the flask (i.e., over the course of about thirty minutes). The reaction mixture was stirred at 0° C. for additional thirty minutes. The excess hydrochloric acid in the reaction mixture was carefully neutralized by addition of solid sodium bicarbonate, and the water was removed using a vacuum rotary evaporator. The solid was extracted with 100 mL of warm (40° C.) methanol. The solution was filtered, and the methanol was removed under vacuum to give the final product as red crystals. The yield was 1.90 g (87% of theoretical yield).

Analysis. ¹H NMR (MeOD-d4, 300 MHz) δ 1.94 (p, ³J_{H-H}=6.9 Hz, 2H, CH₂,), 2.11 (p, ³J_{H-H}=6.9 Hz, 2H, CH₂), 2.64 (t, ³J_{H-H}=6.0 Hz, 2H, CH₂), 2.95 (t, ³J_{H-H}=6.6 Hz, 2H, CH₂), 3.55 (t, ³J_{H-H}=5.7 Hz, 2H), 3.83 (t, ³J_{H-H}=8.1 Hz, 2H), 4.16 (s, 3H, CH₃O), 6.47 (s, 1H), 6.70 (s, 1H).

### Example 15

### Synthesis of Intermediate 13

Intermediate 13 (sodium 3-(7-methoxy-2,2,4-trimethyl-6-nitroso-3,4-dihydroquinolin-1(2H)-yl)propane-1-sulfonate) was prepared according to the following reaction scheme:

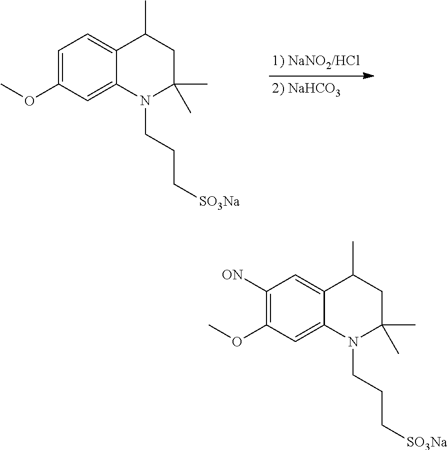

Intermediate 3 (sodium 3-(7-methoxy-2,2,4-trimethyl-3,4-dihydroquinolin-1(2H)-yl)propane-1-sulfonate) (2.00 g, 0.00572 mol) was placed into a 250 mL round-bottomed, one-necked flask equipped with a magnetic stir bar and an addition funnel. A solution of 12 M hydrogen chloride in water (20 mL) was added to the flask. The reaction mixture was cooled using an ice-water bath and a solution of sodium nitrite (0.395 g, 0.00572 mol) in 10 mL of water was slowly added to the flask with stirring. After the addition was completed the stirring was continued for extra 30 minutes at 0° C. The solvent was removed using a vacuum rotary evaporator (the temperature of water bath was set to 40° C. during the evaporation). The residue was dissolved in 20 mL of water and the solution was neutralized using solid sodium bicarbonate. The water was removed under in vacuum to give a yellow solid. The product was extracted from the solid with three portions (50 ml each) of hot methanol. The methanol solutions were combined, filtered and the solvent was removed in vacuum to give a yellow solid product. The yield was 1.80 g (83% of theoretical yield).

Analysis. ¹H NMR (D₂O, 300 MHz) δ 0.96 (d, 3H, ³J_{H-H}=6.9 Hz), 1.17 (t, 1H, ³J_{H-H}=12.6 Hz), 1.22 (s, 3H), 1.34 (s, 3H), 1.7 (m, 1H), 1.9-2.1 (m, 2H), 2.3 (m, 1H), 3.08 (t, 2H, ³J_{H-H}=6.9 Hz), 3.5-3.8 (m, 2H), 4.00 (s, 3H), 5.89 (s, 1H), 6.54 (s, 1H).

### Example 16

### Synthesis of Intermediate 14

The synthesis of intermediate 14 (disodium 4-[7-hydroxy-2,2-dimethyl-6-nitroso-4-(sulfonatomethyl)-3,4-dihydroquinolin-1(2H)-yl]butanoate) is depicted in the following reaction scheme:

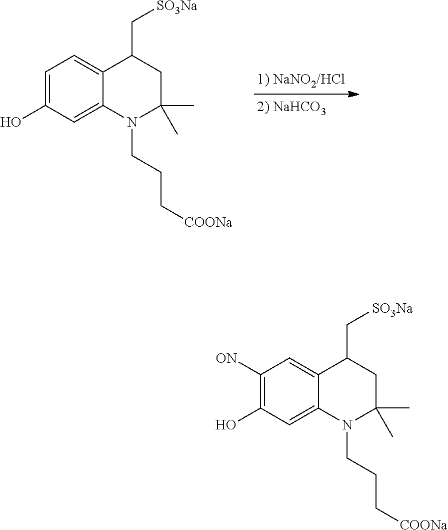

Intermediate 8 (disodium 4-(7-hydroxy-2,2-dimethyl-4-(sulfonatomethyl)-3,4-dihydroquinolin-1(2H)-yl)butanoate) (10.0 g, 0.0280 mol) was placed into a 250 mL round-bottomed, one-necked flask equipped with a magnetic stir bar and an addition funnel. A 12.1 M solution of hydrogen chloride in water (50 mL) and 50 ml of pure water were added to the flask. The reaction mixture was cooled by placing the flask in an ice-water bath. A solution of sodium nitrite (1.94 g, 0.0280 mol) in 30 mL of water was slowly added to the flask with stirring. After the addition was completed the stirring was continued for additional 30 minutes at 0° C. The solvent was removed using a vacuum rotary evaporator (the temperature of water bath was set to 40° C. during the evaporation). The residue was dissolved in 20 mL of water and the solution was neutralized using solid sodium bicarbonate. The solvent was removed under vacuum to give a yellow solid. The product was extracted from the solid using a Soxhlet extractor and methanol as a solvent. Finally, the methanol solution that contained the product was evaporated under in vacuum to give 8.8 g of the final product as a yellow solid.

Analysis. ¹H NMR (D₂O, 300 MHz) δ 1.52 (s, 3H, CH₃), 1.60 (s, 3H, CH₃), 1.8-2.2 (m, 3H), 2.3-2.6 (m, 3H), 3.0-3.4 (m, 2H), 3.5-4.0 (m, 3H), 6.84 (s, 1H) 7.00 (s, 1H).

### Example 17

### Synthesis of Intermediate 16

Intermediate 16 (disodium 4-{7-hydroxy-2,2-dimethyl-6-[(E)-(4-nitrophenyl)diazenyl]-4-(sulfonatomethyl)-3,4-dihydroquinolin-1(2H)-yl}butanoate) was prepared according to the following reaction scheme:

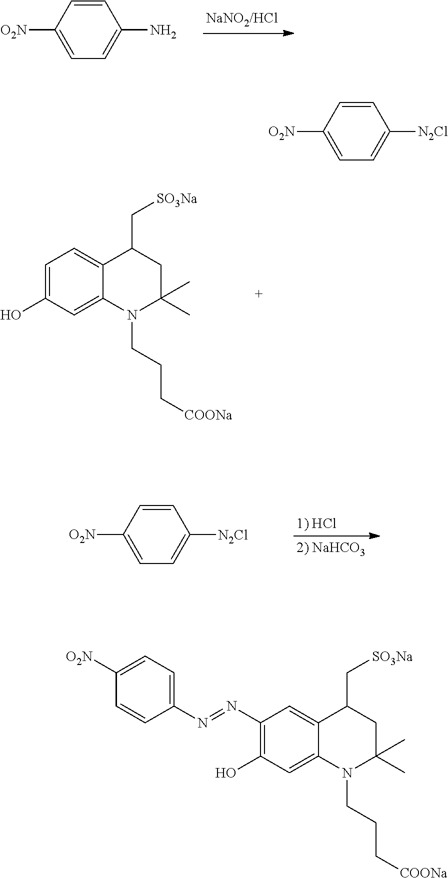

Step 1. p-Nitroaniline (0.546 g, 0.00395 mol) was added to a 100 mL round-bottomed, one-necked flask equipped with a magnetic stir bar and an addition funnel. Ten mL of 10% HCl was added to the flask. The mixture was stirred at room temperature until a clear solution formed, and then the flask was immersed in an ice-water bath. A solution of sodium nitrite (0.273 g, 0.00395 mol) in 5 mL of water was added drop-wise to the flask with stirring. When the addition was complete, the mixture was stirred for another 30 minutes at 0° C.

Step 2. Intermediate 8 (disodium 4-[7-hydroxy-2,2-dimethyl-4-(sulfonatomethyl)-3,4-dihydroquinolin-1(2H)-yl]butanoate) (1.59 g, 0.00395 mol) was added to a 250 mL conical flask, followed by 10 mL of 10% HCl. The flask was immersed in an ice-water cooling bath and stirring was started. The solution of p-nitrophenyldiazonium salt (from step 1) was added to the flask in small portions (1-2 mL) over the course of 10 minutes. The reaction mixture was stirred for 1 hour at 0° C. The precipitated red solid was filtered, washed with water (3×15 mL), and dried (initially in air and then using a vacuum pump overnight). The yield was 1.50 g (75% of theoretical).

Because this zwitterion (1-(3-carboxypropyl)-7-hydroxy-2,2-dimethyl-6-[(E)-(4-nitrophenyl)diazenyl]-1,2,3,4-tetrahydroquinolinium-4-yl)methanesulfonate is practically insoluble in most of the common solvents for characterization it was converted into the sodium salt. For this, 400 mg of the zwitterion was placed in a 250 mL round-bottomed, one-necked flask equipped with a magnetic stirrer. Water (100 mL) was added to the flask and the stirring was started. Solid sodium bicarbonate (300 mg) was added to the flask in five portions to avoid excessive foaming. The solvent was removed using a rotary evaporator. The solid residue was re-dissolved in methanol. The solution was filtered to remove impurities (mostly undissolved sodium bicarbonate) and the methanol was removed using a rotary evaporator. The residue was re-dissolved in 25 mL of water, and the volume was reduced to 10 mL using the rotary evaporator (this operation was necessary to remove traces of methanol). The solution was frozen and lyophilized overnight. The yield of red solid product was 367 mg (66% total yield).

Analysis. ¹H NMR (dmso-d6, 300 MHz) δ 1.20 (s, 3H, CH₃), 1.32 (s, 3H, CH₃), 1.44 (t, 1H, ³J_{H-H}=13.2 Hz), 1.70 (m, 2H), 2.11 (m, 2H), 2.34 (d, 1H, ³J_{H-H}=13.2 Hz), 2.49 (d, 1H, ³J_{H-H}=7.2 Hz), 3.04 (m, 2H), 3.10 (d, 1H, ³J_{H-H}=13.2 Hz), 3.26 (m, 1H), 3.43 (m, 1H), 5.98 (s, 1H), 7.27 (s, 1H), 7.70 (d, 2H, ³J_{H-H}=9.0 Hz), 8.19 (d, 2H, ³J_{H-H}=9.0 Hz). MS (ES)=505.5 (M), theoretical 505.2.

### Example 18

### Synthesis of Intermediate 17

The following reaction scheme illustrates the synthesis of intermediate 17 ((E)-(1-(3-carboxypropyl)-2,2-dimethyl-6-((4-nitro phenyl)diazenyl)-1,2-dihydroquinolinium-4-yl)methanesulfonate):

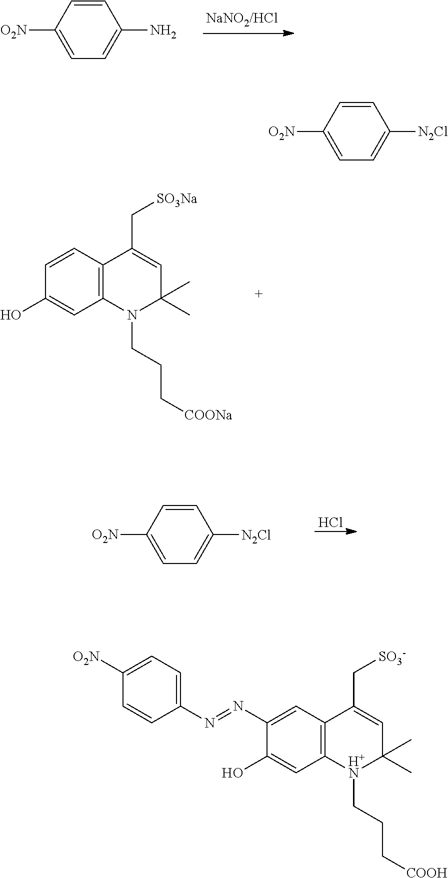

Step 1. p-Nitroaniline (0.546 g, 0.00395 mol) was added to a 100 mL round-bottomed, one-necked flask equipped with a magnetic stir bar and an addition funnel. Then 10 mL of 10% HCl was added to the flask. The mixture was stirred at room temperature until a clear solution formed, and then the flask was immersed in an ice-water bath. A solution of sodium nitrite (0.273 g, 0.00395 mol) in 5 mL of water was added drop-wise to the flask with stirring. When the addition was complete, the mixture was stirred for another 30 minutes at 0° C.

Step 2. Intermediate 10 (disodium 4-(7-hydroxy-2,2-dimethyl-4-(sulfonatomethyl)quinolin-1(2H)-yl)butanoate) (1.58 g, 0.00395 mol) was placed in a 250 mL conical flask, followed by 10 mL of 10% HCl. The flask was immersed in an ice-water cooling bath and stirring was started. The solution of p-nitrophenyldiazonium salt (from step 1) was added to the flask in small portions (1-2 mL) over the course of about 10 minutes. The reaction mixture was stirred for 1 hour at 0° C. The precipitated red solid was filtered, washed with water (3×15 mL), and dried (initially in air and then using a high vacuum pump overnight). The yield of the red solid product was 1.40 g (70% of theoretical yield). Because the product is almost insoluble in most major common solvents, the NMR sample was obtained from a diisopropylethylammonium salt.

Analysis. ¹H NMR (dmso-d6, 300 MHz) δ 1.41 (s, 6H, 2*CH₃), 1.84 (p, 2H, ³J_{H-H}=6.6 Hz, CH₂CH₂CH₂CO₂), 2.34 (t, 2H, ³J_{H-H}=7.2 Hz, CH₂CO₂), 3.50 (distorted t, 2H, CH₂N), 3.56 (s, 2H, CH₂SO₃), 5.79 (s, 1H, CH), 5.85 (s, 1H), 7.24 (s, 1H) 7.65 (d, 1H, ³J_{H-H}=9.0 Hz), 8.22 (d, 1H, ³J_{H-H}=9.0 Hz). MS=503.6 (M), theoretical mass=503.1;

### Example 19

### Synthesis of Dye 2 Carboxylic Acid

Dye 2 (sodium 3-(11-(3-carboxypropyl)-10,10-dimethyl-8-(sulfonatomethyl)-2,3,4,8,9,10-hexahydro-1H-dipyrido[3,2-b:2′,3′-i]phenoxazin-11-ium-1-yl)propane-1-sulfonate) was prepared by reacting intermediate 14 and intermediate 1 according to the following reaction scheme:

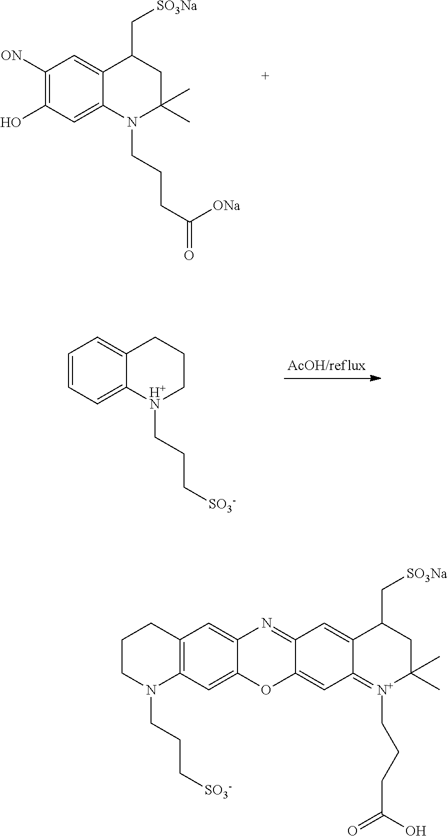

Intermediate 14 ([1-(3-carboxypropyl)-7-hydroxy-2,2-dimethyl-6-nitroso-1,2,3,4-tetrahydroquinolinium-4-yl]methanesulfonate) (1.00 g, 0.00259 mol), intermediate 1 (3-(3,4-dihydroquinolinium-1(2H)-yl)propane-1-sulfonate) (0.662 g, 0.00259 mol), sodium acetate (0.426 g, 0.00519 mol), and acetic acid (25 mL, 0.44 mol) were placed into a 100 mL round-bottomed flask attached to a reflux condenser. The reaction mixture was stirred under reflux for 2 hours. During this time, the reaction mixture became deep-blue in color. TLC showed a complete consumption of the starting material and formation of the new deeply blue-colored product. The acetic acid was removed under vacuum. The residue was purified using preparative reverse-phase (RP) HPLC. The C18 reverse phase column (ModCol column) was 50×250 mm, and the program comprised 60 minutes with 100% water, 120 minutes with 10% ACN/90% water, and 100 minutes with 70% ACN/30% water, with an elution rate of 25 mL/min. The product eluted at 140-160 minutes (total time). The dye-containing fractions were combined and the solvent was removed under vacuum. The residue was dissolved in 10 mL of deionzed water, frozen, and lyophilized. The yield of the final product was 0.036 g (2.2% of theoretical yield).

Analysis. ¹H NMR (D₂O, 300 MHz) δ 1.50 (s, 3H, CH₃), 1.64 (s, 3H, CH₃), 1.8 (t, 1H, ³J_{H-H}=7.0 Hz), 1.9-2.1 (m, 2H, CH₂), 2.6-2.8 (m, 3H), 2.9-3.1 (m, 1H), 3.5-4.0 (m, 4H), 7.06 (s, 1H), 7.80 (s, 1H). MS (M⁻)=620.

### Example 20

### Synthesis of Dye 3 Carboxylic Acid

The following reaction scheme depicts the synthesis of Dye 3 (sodium 3-(11-(3-carboxypropyl)-2,2,4,10,10-pentamethyl-8-(sulfonatomethyl)-2,3,4,8,9,10-hexahydro-1H-dipyrido[3,2-b:2′,3′-i]phenoxazin-11-ium-1-yl)propane-1-sulfonate):

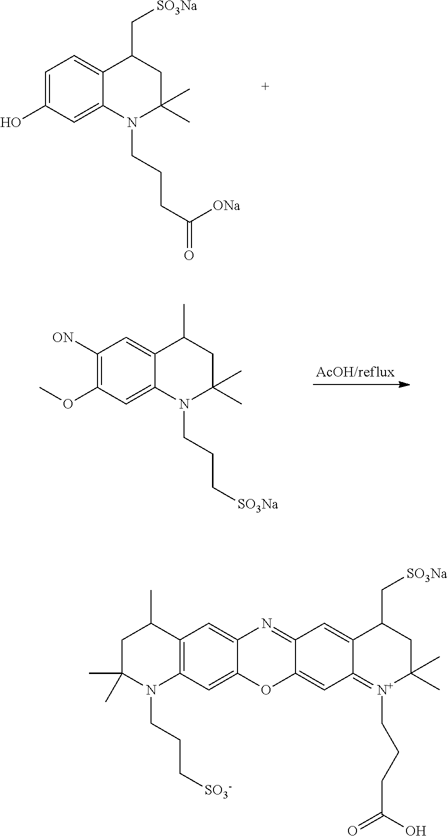

Intermediate 8 (disodium 4-[7-hydroxy-2,2-dimethyl-4-(sulfonatomethyl)-3,4-dihydroquinolin-1(2H)-yl]butanoate) (600 mg, 0.001 mol), intermediate 13 (sodium 3-(7-methoxy-2,2,4-trimethyl-6-nitroso-3,4-dihydroquinolin-1(2H)-yl)propane-1-sulfonate) (566 mg, 0.00149 mol), and acetic acid (25 mL, 0.44 mol) were added to a 100 mL round-bottomed, one-necked flask set up with a magnetic stir bar and a reflux condenser. The reaction mixture was stirred with heating at reflux for 2 hours. During this time, the reaction mixture became dark blue in color. TLC showed the disappearance of the starting material and formation of the blue-colored reaction product (SiO₂, eluent: acetone-water (1:1), R_{f} starting material 0 and 0.6, R_{f} of the product 0.1). The solvent was removed using vacuum and the residue was dissolved in 50 mL of acetone-water mixture (4:1). The solution was mixed with 10 g of silica gel and the solvent was removed using vacuum. The silica/dye mixture was loaded onto a silica gel column, and the column was rinsed first with acetone (1000 mL) and the dye was eluted with acetone/water (10:1). The fractions containing the product were combined and evaporated. The yield of the dye was 700 mg (70% of theoretical yield). HPLC analysis showed a presence of about 10% of a closely eluting impurity. The dye was additionally purified on preparative reverse-phase HPLC (as described above in Example 19. The product eluted at 150-160 minutes (total time). The yield of pure dye was 180 mg (20% of theoretical yield).

Analysis. ¹H NMR (MeOD, 300 MHz) δ 1.47 (s, 6H), 1.48 (s, 3H), 1.56 (s, 6H), 1.67 (t, 1H, ³J_{H-H}=12.6 Hz), 1.81 (t, 1H, ³J_{H-H}=12.6 Hz), 2.00 (m, 3H), 2.2 (m, 2H), 2.6 (m, 3H), 3.0 (m, 4H), 3.3-4.0 (m, 6H), 7.09 (s, 1H), 7.11 (s, 1H), 7.62 (s, 1H), 7.82 (s, 1H). MS (M⁻)=662.

### Example 21

### Synthesis of Dye 3 Maleimide

Dye 3 maleimide (sodium 3-(11-(4-(2-(2,5-dioxo-2,5-dihydro-1H-pyrrol-1-yl)ethylamino)-4-oxobutyl)-2,2,4,10,10-pentamethyl-8-(sulfonatomethyl)-2,3,4,8,9,10-hexahydro-1H-dipyrido[3,2-b:2′,3′-i]phenoxazin-11-ium-1-yl)propane-1-sulfonate) was prepared according to the following reaction scheme:

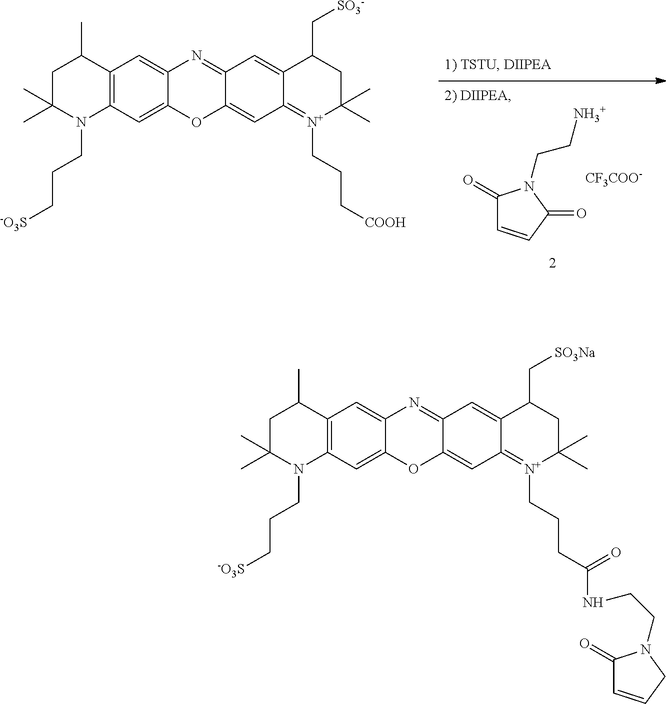

A 100 mL round-bottom flask was charged with dye 3 (sodium 3-(11-(3-carboxypropyl)-2,2,4,10,10-pentamethyl-8-(sulfonatomethyl)-2,3,4,8,9,10-hexahydro-1H-dipyrido[3,2-b:2′,3′-i]phenoxazin-11-ium-1-yl)propane-1-sulfonate) (0.05 g, 0.00007 mol), N,N-dimethylformamide (10 mL, 0.1 mol), and N,N-diisopropylethylamine (0.0292 mL, 0.000168 mol). The mixture was stirred and O-(N-succinimidyl)-N,N,N′,N′-tetramethyluronium tetrafluoroborate (0.0263 g, 0.0000875 mol) was added to the reaction mixture in one portion. The flask was flushed with nitrogen and stirring was continued for 30 min under nitrogen. Then 2-(2,5-dioxo-2,5-dihydro-pyrrol-1-yl)-ethyl-ammonium trifluoroacetate (0.020 g, 0.000080 mol) was added to the flask in one portion and the reaction mixture was stirred at room temperature for 2 hours. The solvent was removed using a vacuum pump (water bath temperature was set to 30° C.). The residue was dissolved in solution of 2 g of sodium perchlorate in 25 mL of water. The product was purified on a preparative RP HPLC column, which was eluted with water and acetonitrile. (Program: 0-60 min 100% water, 60-120 min 10% acetonitrile/90% water, 120-150 min 50% acetonitrile/50% water, 150-180 min 75% acetonitrile/25% water, 180-200 min 100% water.) The dye was eluted at 95-115 min. The dye-containing fractions were combined, and the solvent was reduced from about 100 to 10 mL using a rotary evaporator (water bath was set to 30° C.). The solution was frozen and lyophilized overnight. The yield of the final product (blue solid) was 20.0 mg (30% of theoretical yield).

Analysis. ¹H NMR (MeOD-d4, 300 MHz) δ 1.47-1.50 (m, 9H, 3*CH₃), 1.56 (s, 3H, CH₃), 1.57 (s, 3H, CH₃), 1.70 (t, 1H, ³J_{H-H}=12.9 Hz), 1.80 (t, 1H, ³J_{H-H}=12.9 Hz), 1.9-2.1 (m, 3H), 2.2-2.4 (m, 4H), 2.6-2.7 (m, 2H), 2.9-3.1 (m, 5H), 3.3-4.0 (m, 12H), 6.80 (s, 2H), 7.12 (s, 1H), 7.13 (s, 1H), 7.65 (s, 1H), 7.85 (s, 1H).

### Example 22

### Synthesis of Dye 6 Carboxylic Acid

The following reaction scheme depicts the preparation of Dye 6 (sodium (1,1′-bis(3-carboxypropyl)-2,2,10,10-tetramethyl-2,3,4,8,9,10-hexahydro-1H-dipyrido[3,2-b:2′,3′-i]phenoxazine-11-ium-4,8-diyl)dimethanesulfonate):

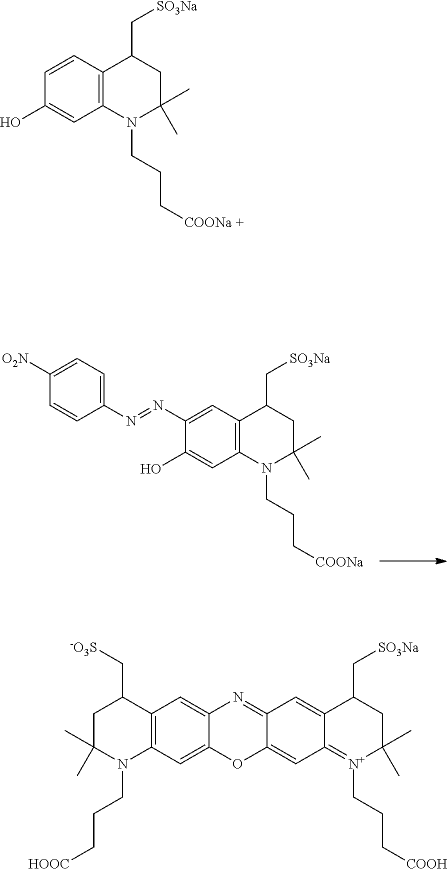

Intermediate 16 ((E)-(7-hydroxy-1-(3-carboxypropyl)-2,2-dimethyl-6-((4-nitrophenyl)diazenyl)-1,2,3,4-tetrahydroquinolinium-4-yl)methanesulfonate) (0.671 g, 0.00132 mol), intermediate 8 (disodium 4-[7-hydroxy-2,2-dimethyl-4-(sulfonatomethyl)-3,4-dihydroquinolin-1(2H)-yl]butanoate) (0.532 g, 0.00132 mol), and acetic acid (25 mL, 0.44 mol) were added to a 100 mL round-bottomed, one-necked flask set up with a magnetic stir bar and a reflux condenser. The reaction mixture was heated at reflux for 24 hours. TLC showed complete consumption of starting material and formation of deeply blue-colored product (SiO₂, eluent: acetone-water (1:1), R_{f} product=0.1, R_{f} starting material=0, and 0.4). The reaction mixture was evaporated and the residue was dissolved in 50 mL of a mixture of acetone-water (4:1). The solution was mixed with 10 g silica and the solvent was removed under vacuum. The dye/silica mixture was loaded onto a silica column prepared in acetone (diameter×length=1 in.×2 in.). The column was eluted with an acetone-water gradient (0-10%). The dye containing fractions were combined and the solvent was removed using a rotary evaporator. NMR analysis showed the presence of impurities, and the dye was further purified on a preparative RP HPLC column, which was eluted with water and acetonitrile. (Program: 0-60 min 100% water, 60-120 min 10% acetonitrile/90% water, 120-150 min 50% acetonitrile/50% water, 150-180 min 80% acetonitrile/20% water, 180-200 min 100% water.) The dye eluted at 105-115 min. The dye-containing fractions were combined and reduced in volume from about 120 mL to 10 mL using a rotary evaporator. The dye solution was frozen and lyophilized. The yield of pure dye (blue solid) was 100 mg (10% of theoretical yield).

Analysis. ¹H NMR (D₂O, 300 MHz) δ 1.47 (s, 6H, 2*CH₃), 1.57 (s, 6H, 2*CH₃), 1.81 (t, 1H, ³J_{H-H}=12.9 Hz), 2.02 (m, 4H), 2.40 (m, 2H), 2.55 (t, 4H, ³J_{H-H}=6.0 Hz), 2.67 (dd, 2H, ³J_{H-H}=12.9 Hz, ⁴J_{H-H}=3.0), 3.0 (m, 2H), 3.3-3.9 (m, 6H), 7.11 (s, 2H), 7.82 (s, 2H). MS=708.8 (positive ion detection, M-Na⁺ +2H⁺), theoretical mass 708.2.

### Example 23

### Synthesis of Dye 7 Carboxylic Acid

Dye 7 (sodium (1,1′-bis(3-carboxypropyl)-2,2,10,10-tetramethyl-2,10-dihydro-1H-dipyrido[3,2-b:2′,3′-i]ph enoxazine-11-ium-4,8-diyl)dimethanesulfonate) was prepared in accordance with the following reaction scheme:

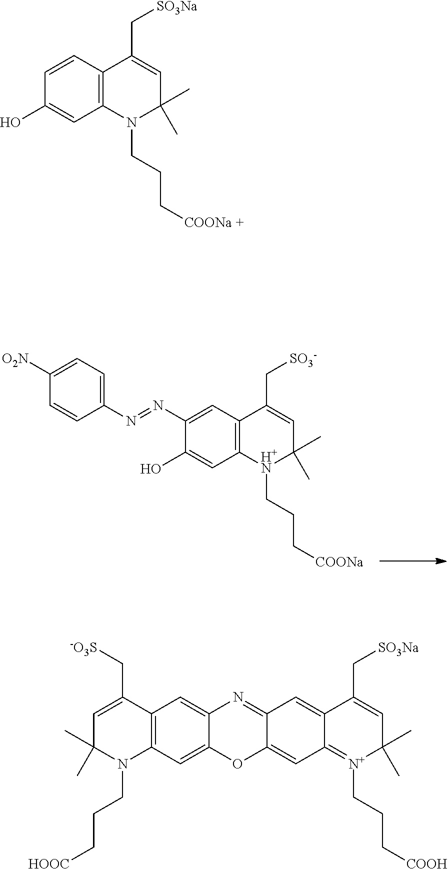

Intermediate 17 ((E)-(1-(3-carboxypropyl)-2,2-dimethyl-6-((4-nitrophenyl)diazenyl)-1,2-dihydroquinolinium-4-yl)methanesulfonate) (399 mg, 0.000791 mol), intermediate 10 (disodium 4-(7-hydroxy-2,2-dimethyl-4-(sulfonatomethyl)quinolin-1(2H)-yl)butanoate) (332 mg, 0.000830 mol), acetic acid (15 mL, 0.26 mol), and sodium acetate (80.0 mg, 0.000975 mol) were added a 100 mL round-bottomed, one-necked flask set up with a magnetic stir bar and a reflux condenser. The mixture was heated at gentle reflux with stirring for 6 hours. TLC showed complete consumption of the starting material and formation of the deeply blue-colored product. (SiO₂, eluent: acetone-water (1:1), R_{f} product=0.1, R_{f} starting material=0, and 0.4). The acetic acid was removed under vacuum and the residue was purified by column chromatography on SiO₂ (1×2 inch column) using 2:1 acetone-water mixture as eluent (TLC: acetone-water 1:3, R_{f}=0.1). The blue, dye-containing fraction was collected. Removal of the solvent produced a blue solid; the weight was 250 mg. The isolated compound was not pure based on ¹H NMR and HPLC analysis. The product was additionally purified using preparative RP HPLC (ModCol column, 50×250 mm; program: 0-60 min 100% water, 60-120 min 10% Acetonitrile/90% water, 120-160 min 50% Acetonitrile/50% water, 160-220 min 80% Acetonitrile/20% water, 220-240 min 100% water). The dye-containing fraction (green-blue) was eluted at 90-95 min. The fraction was collected and reduced in volume (from about 100 mL to about 10 mL) using a rotary evaporator (the water bath temperature was set to 40° C.). The solution was frozen and lyophilized. The yield of pure dye (blue solid) was 17 mg (3% of theoretical yield).

Analysis. ¹H NMR (D₂O, 300 MHz) δ 1.60 (s, 12H, 4*CH₃), 1.80-2.00 (m, 4H), 2.30-2.50 (m, 4H), 3.70-3.90 (m, 4H), 3.9-4.1 (m, 4H), 6.03 (s, 2H), 7.79 (s, 2H). MS=702.6 (negative ion detection, M⁻), theoretical mass 702.2.

### Example 24

### Synthesis of Intermediate 18

The following reaction scheme illustrates the synthesis of intermediate 18, disodium 3-(7-methoxy-2,2-dimethyl-4-(sulfonatomethyl)quinolin-1(2H)-yl)propane-1-sulfonate:

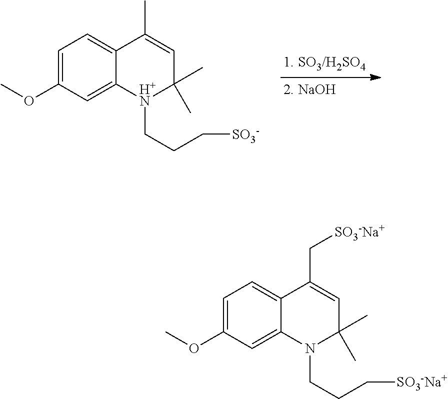

Sulfuric acid (50.0 mL, 0.938 mol) was added to a 250 mL round-bottom three-necked flask equipped with a thermometer, additional funnel, nitrogen inlet, a and magnetic stir bar. The flask was immersed in ice-salt cooling bath and stirring was started. Intermediate 2,3-(7-methoxy-2,2,4-trimethylquinolinium-1(2H)-yl)propane-1-sulfonate (25.00 g, 0.07682 mol) was slowly added to the flask by about 2 g portions during 20 minutes with stirring. At the end of the addition, the reaction mixture become yellow-colored. A 7.2 M solution of sulfur trioxide in sulfuric acid (14.0 mL) was placed in dropping funnel. The solution was slowly added to the reaction mixture at such rate that temperature inside the flask didn't rise higher than 5° C. When the addition was completed, the cooling bath was removed and the reaction mixture was stirred at room temperature for 72 hours. The reaction mixture was poured in 1 L beaker containing 100 g of crushed ice. The beaker was immersed in ice-water cooling bath. The acid was neutralized by addition of a cold solution of sodium hydroxide in water. The pH of the reaction mixture at the end of neutralization was about 9. The beaker was left in the cooling bath for 30 min. Sodium sulfate hydrate that precipitated out of solution was removed by filtration, and the filtrate was evaporated using a rotovap. The residual solid (the product and some sodium sulfate) was extracted with hot ethanol (3×200 mL). Combined ethanol extracts were evaporated using a rotovap to give final product as a white solid. The yield was 26.0 g (76%, theoretical).

Analysis. ¹H NMR (D₂O, 300 MHz) δ 1.25 (s, 6H, 2×CH₃), 1.93 (p, ³J_{H-H}=7.5 Hz, 2H), 2.93 (t, ³J_{H-H}=7.5 Hz, 2H), 3.32 (distorted t, ³J_{H-H}=7.5 Hz, 1H), 3.75 (s, 3H), 3.87 (s, 2H), 5.51 (s, 1H), 6.19 (s, 1H, ⁴J_{H-H}=2.4 Hz), 6.29 (dd, 1H, ³J_{H-H}=8.7 Hz, ⁴J_{H-H}=2.4 Hz), 7.20 (d, 1H, ³J_{H-H}=8.7 Hz).

### Example 25

### Synthesis of Intermediate 19

The following reaction scheme illustrates the synthesis of intermediate 19, disodium 3-(7-methoxy-2,2-dimethyl-4-(sulfonatomethyl)-3,4-dihydroquinolin-1(2H)-yl)propane-1-sulfonate:

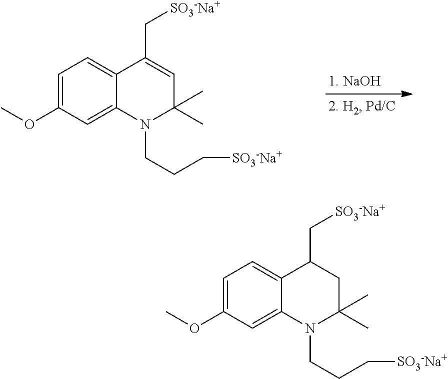

Intermediate 18, disodium 3-[7-methoxy-2,2-dimethyl-4-(sulfonatomethyl)quinolin-1(2H)-yl]propane-1-sulfonate (24 g, 0.052 mol) was added to a 1 L Parr hydrogenation bottle followed by 150 mL of deionized water and Pd on carbon (10%, 1.00 g). The bottle was attached to a high-pressure Parr hydrogenation apparatus and the system was evacuated using a water aspirator and then filled with hydrogen gas. The evacuation/filling procedure was repeated three more times. Then the system was filled with hydrogen at 50 psi pressure. The bottle was shaken at room temperature for 4 hours until hydrogen consumption stopped. The remaining pressure was carefully released and the catalyst was removed by vacuum filtration through a Celite pad. The removal of solvent in vacuum gave the final product as a white solid. The yield was 20 g (93%, theoretical).

Analysis: ¹H NMR (D₂O, 300 MHz) δ 1.08 (s, 3H), 1.27 (s, 3H), 1.54 (t, 1H, ²J_{H-H}=12.6 Hz), 1.9-2.1 (m, 2H), 2.20 (dd, 1H, ²J_{H-H}=12.6 Hz, ³J_{H-H}=5.4 Hz), 2.8-2.95 (m, 3H), 3.2-3.3 (m, 2H), 3.4-3.55 (m, 2H), 3.72 (s, 3H), 6.22 (s, 1H, ⁴J_{H-H}=2.4 Hz), 6.31 (dd, 1H, ³J_{H-H}=8.7 Hz, ⁴J_{H-H}=2.4 Hz), 7.12 (d, 1H, ³J_{H-H}=8.7 Hz).

### Example 26

### Synthesis of Intermediate 20

The following reaction scheme illustrates the synthesis of intermediate 20. disodium (E)-3-(7-methoxy-2,2-dimethyl-6-((4-nitrophenyl)diazenyl)-4-(sulfonatomethyl)-3,4-dihydroquinolin-1(2H)-yl)propane-1-sulfonate:

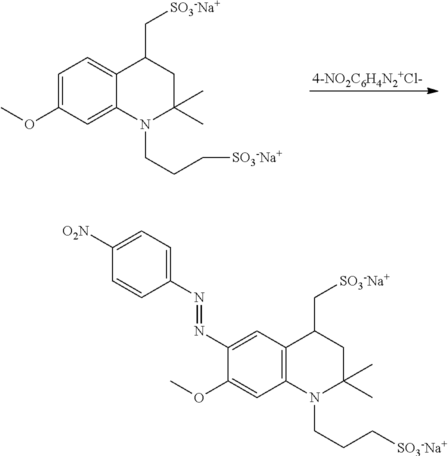

Step 1. p-Nitroaniline (1.66 g, 0.0120 mol) was added to a 100 mL round-bottomed, one-necked flask equipped with a magnetic stir bar and an addition funnel. 20 mL of 10% HCl was added to the flask. The mixture was stirred at room temperature until a clear solution formed, and then the flask was immersed in an ice-water bath. A solution of sodium nitrite (0.827 g, 0.0120 mol) in 10 mL of water was added drop-wise to the flask with stirring. When the addition was complete, the mixture was stirred for another 30 minutes at 0° C.

Step 2. Intermediate 19, disodium 3-[7-methoxy-2,2-dimethyl-4-(sulfonatomethyl)quinolin-1(2H)-yl]propane-1-sulfonate (5.41 g, 0.0120 mol), was added to a 250 mL conical flask, followed by 40 mL of 10% HCl. The flask was immersed in an ice-water cooling bath and stirring was started. The solution of the p-nitrophenyldiazonium salt (from step 1) was added to the reaction mixture by small portions (1-2 mL) over the course of 10 minutes. The reaction mixture was stirred for 1 hour at 0° C. The reaction mixture was neutralized with solid sodium bicarbonate. The solvent was removed. The orange residue was extracted with boiling ethanol (3×150 mL). The combined ethanol extracts were evaporated using a rotovap. The product (orange solid) was dried under vacuum. The yield was 5.01 g (70%, theoretical) of orange solid.

Analysis. ¹H NMR (MeOD, 300 MHz) δ 1.33 (s, 3H), 1.47 (s, 3H), 1.64 (t, 1H, ³J_{H-H}=12.6 Hz), 2.05-2.25 (m, 2H), 2.59 (dd, 1H, ³J_{H-H}=12.6 and 4.8 Hz), 2.8-3.0 (m, 3H), 3.2-3.3 (m, 1H), 3.55-3.80 (m, 3H), 4.06 (s, 3H, CH₃O) 6.44 (bs, 1H), 7.26 (s, 1H), 7.82 (s, 1H), 7.89 (d, ³J_{H-H}=9.0 Hz), 8.30 (d, ³J_{H-H}=9.0 Hz).

### Example 27

### Synthesis of Dye 14 Carboxylic Acid

The following reaction scheme illustrates the synthesis of Dye 14 carboxylic acid disodium (11-(3-carboxypropyl)-2,2,10,10-tetramethyl-1-(3-sulfonatopropyl)-2,3,4,8,9,10-hexahydro-1H-dipyrido[3,2-b:2′,3′-i]phenoxazine-11-ium-4,8-diyl)dimethanesulfonate:

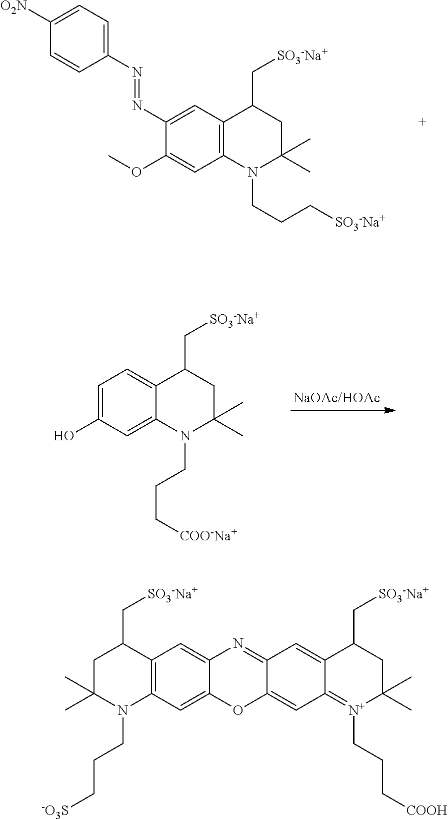

Intermediate 19 (disodium 3-[6-[(E)-(4-nitrophenyl)diazenyl]-7-methoxy-2,2-dimethyl-4-(sulfonatomethyl)-3,4-dihydroquinolin-1(2H)-yl]propane-1-sulfonate) (1.15 g, 0.00199 mol), intermediate 8 (disodium 4-[7-hydroxy-2,2-dimethyl-4-(sulfonatomethyl)-3,4-dihydroquinolin-1(2H)-yl]butanoate) (0.960 g, 0.00239 mol), acetic acid (25 mL, 0.44 mol), sodium acetate (1.00 g, 0.0122 mol), acetic acid (2.5 mL, 0.044 mol), and deionized water (50 mL) were added to a 250 mL round-bottomed, one-necked flask set up with a magnetic stir bar and a reflux condenser. The reaction mixture was heated at gentle reflux under nitrogen for 3 hours. The color of the reaction mixture changed from orange-red to deep blue. TLC showed complete consumption of starting material and formation of deeply blue-colored product (SiO₂ eluent: acetone-water (1:2), R_{f} product=0.1, R_{f} starting material=0 and 0.2). The reaction mixture was cooled to room temperature and solvents were removed using a rotovap. The blue solid was dissolved in 50 mL of acetone-water (1:2) mixture. The solution was loaded on Versa-Flash SiO₂ cartridge prewashed with acetone. The dye was eluted from the cartridge using acetone-water mixtures. The dye-containing fractions were combined and solvent was evaporated using a rotovap. A blue residue was suspended in pure acetone. The product was filtered and dried. The yield was 0.850 g as a blue solid (54%, theoretical).

Analysis. ¹H NMR (MeOD, 300 MHz) δ 1.48 (s, 6H), 1.58 (s, 6H), 1.81 (t, 2H, ³J_{H-H}=12.6 Hz), 2.00 (m, 3H), 2.2 (m, 2H), 2.3 (m, 1H), 2.6 (m, 3H), 3.0 (m, 4H), 3.3-4.1 (m, 8H), 7.04 (bs, 1H), 7.26 (bs, 1H), 7.83 (m, 2H). MS (MH⁻)=742, (M²⁻/2)=371.

### Example 28

### Synthesis of Dye 14 Succinimidyl Ester

The following reaction scheme illustrates the synthesis of Dye 14 succinimidyl ester, di(ethyl-diisopropyl-ammonium) (11-(4-(2,5-dioxopyrrolidin-1-yloxy)-4-oxobutyl)-2,2,10,10-tetramethyl-1-(3-sulfonatopropyl)-2,3,4,8,9,10-hexahydro-1H-dipyrido[3,2-b:2′,3′-i]phenoxazine-11-ium-4,8-diyl)dimethanesulfonate:

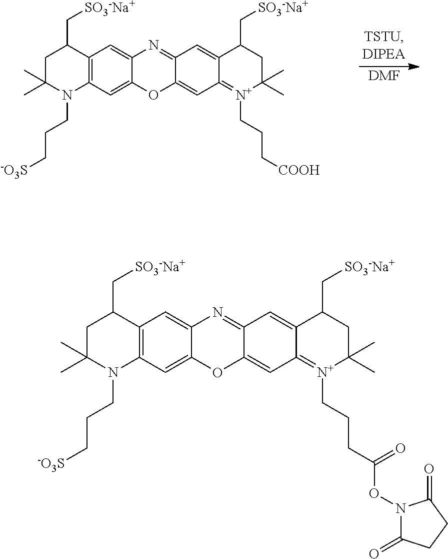

A 25 mL round-bottom one-necked flask was charged with dye 14, disodium (11-(3-carboxypropyl)-2,2,10,10-tetramethyl-1-(3-sulfonatopropyl)-2,3,4,8,9,10-hexahydro-1H-dipyrido[3,2-b:2′,3′-i]phenoxazine-11-ium-4,8-diyl)dimethanesulfonate (0.050 g, 0.000063 mol), O-(N-succinimidyl)-N,N,N′,N′-tetramethyluronium tetrafluoroborate (21.0 mg, 0.00007 mol), and 1 mL of N,N-dimethylformamide. The flask was flushed with nitrogen. A solution of N,N-diisopropylethylamine (10.4 mg, 0.0000802 mol) in 1.00 mL of dimethylformamide was added to the reaction mixture. The flask was closed with a stopper and the reaction mixture was stirred at room temperature for 2 hours. The solvent was removed using a rotovap and oil vacuum pump. The blue solid was dissolved in 5.0 mL of cold water. The solution was filtered through a glass wool plug, frozen and lyophilized for 48 hours. The yield of blue solid was 60 mg (100%, theoretical). Analysis of the solid by NMR and HPLC-MS revealed that the purity was greater than 98%.

Analysis. ¹H NMR (MeOD, 300 MHz) δ 1.30-1.45 (m, 30H), 1.48 (m, 6H), 1.56 (s, 6H), 1.81 (t, 2H, ³J_{H-H}=12.6 Hz), 2.00 (m, 3H), 2.2 (m, 2H), 2.3 (m, 2H), 2.6 (m, 5H), 2.8 (s, 4H), 3.0 (m, 8H), 3.3-4.0 (m, 12H), 7.06 (s, 1H), 7.11 (s, 1H), 7.64 (s, 1H), 7.84 (s, 1H). MS (negative ions): 838 (M⁻), 419 (M²⁻).

### Example 29

### Synthesis of Dye 2 Succinimidyl Ester

The following reaction scheme illustrates the synthesis of Dye 2 succinimidyl ester, ethyl-diisopropyl-ammonium 3-(11-(4-(2,5-dioxopyrrolidin-1-yloxy)-4-oxobutyl)-10,10-dimethyl-8-(sulfonatomethyl)-2,3,4,8,9,10-hexahydro-1H-dipyrido[3,2-b:2′,3′-i]phenoxazin-11-ium-1-yl)propane-1-sulfonate:

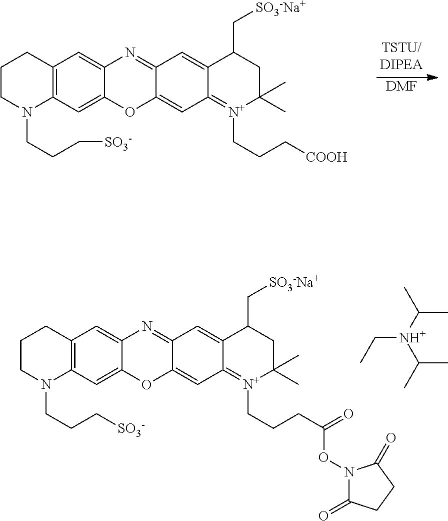

A 25 mL round-bottom one-necked flask was charged with Dye 2, sodium 3-(11-(3-carboxypropyl)-10,10-dimethyl-8-(sulfonatomethyl)-2,3,4,8,9,10-hexahydro-1H-dipyrido[3,2-b:2′,3′-i]phenoxazin-11-ium-1-yl)propane-1-sulfonate (0.050 g, 0.000078 mol), O-(N-succinimidyl)-N,N,N′,N′-tetramethyluronium tetrafluoroborate (25.7 mg, 0.000085 mol), and 1 mL of N,N-dimethylformamide. The flask was flushed with nitrogen. A solution of N,N-diisopropylethylamine (10.4 mg, 0.0000802 mol) in 1.00 mL of dimethylformamide was added to the reaction mixture. The flask was closed with a stopper and the reaction mixture was stirred at room temperature for 2 hours. The solvent was removed using a rotovap and oil vacuum pump. The blue solid was dissolved in 5.0 mL of cold water. The solution was filtered through a glass wool plug, frozen and lyophilized for 48 hours. The yield of blue solid was 65 mg (94%, theoretical). Analysis of the solid by NMR and HPLC-MS revealed that the purity was greater than 98%.

Analysis. ¹H NMR (MeOD, 300 MHz) δ 1.30-1.45 (m, 24H), 1.46 (s, 3H), 1.55 (s, 3H), 1.79 (t, 1H, ³J_{H-H}=12.6 Hz), 2.00-2.2 (m, 5H), 2.6 (m, 3H), 2.8 (s, 4H), 3.00-4.0 (m, 10H), 7.06 (s, 1H), 7.11 (s, 1H), 7.64 (s, 1H), 7.84 (s, 1H).

### Example 30

### Synthesis of Dye 3 Succinimidyl Ester

The following reaction scheme illustrates the synthesis of Dye 3 succinimidyl ester, ethyl-diisopropyl-ammonium 3-(11-(4-(2,5-dioxopyrrolidin-1-yloxy)-4-oxobutyl)-2,2,4,10,10-pentamethyl-8-(sulfonatomethyl)-2,3,4,8,9,10-hexahydro-1H-dipyrido[3,2-b:2′,3′-i]phenoxazin-11-ium-1-yl)propane-1-sulfonate:

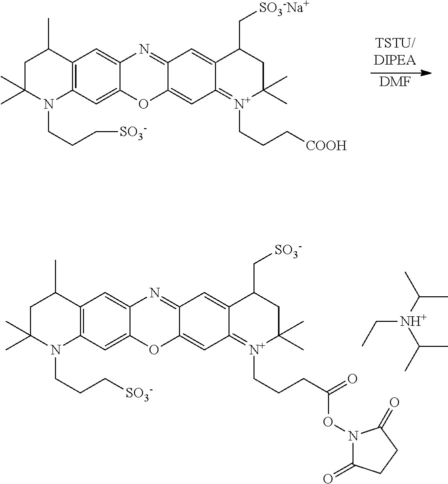

A 25 mL round-bottom one-necked flask was charged with Dye 3, sodium 3-(11-(3-carboxypropyl)-2,2,4,10,10-pentamethyl-8-(sulfonatomethyl)-2,3,4,8,9,10-hexahydro-1H-dipyrido[3,2-b:2′,3′-i]phenoxazin-11-ium-1-yl)propane-1-sulfonate (0.050 g, 0.00007 mol), O-(N-succinimidyl)-N,N,N′,N′-tetramethyluronium tetrafluoroborate (21.9 mg, 0.0000729 mol), and 1 mL of N,N-dimethylformamide. The flask was flushed with nitrogen. A solution of N,N-diisopropylethylamine (11.5 mg, 0.00009 mol) in 1.00 mL of dimethylformamide was added to the reaction mixture. The flask was closed with a stopper and the reaction mixture was stirred at room temperature for 2 hours. The solvent was removed using a rotovap and oil vacuum pump. The blue solid was dissolved in 5.0 mL of cold water. The solution was filtered through a glass wool plug, frozen and lyophilized for 48 hours. The yield of blue solid was 60 mg (92%, theoretical). Analysis of the solid by NMR and HPLC-MS revealed that the purity was greater than 98%.

Analysis. ¹H NMR (MeOD, 300 MHz) δ 1.30-1.45 (m, 15H), 1.48 (m, 9H), 1.56 (s, 6H), 1.67 (t, 1H, ³J_{H-H}=12.6 Hz), 1.81 (t, 1H, ³J_{H-H}=12.6 Hz), 2.00 (m, 3H), 2.2 (m, 2H), 2.6 (m, 3H), 2.8 (s, 4H), 3.0 (m, 4H), 3.3-4.0 (m, 6H), 7.06 (s, 1H), 7.11 (s, 1H), 7.64 (s, 1H), 7.84 (s, 1H).

### Example 31

### Protein Conjugates of Oxazine Dyes

A series of dye conjugates of goat anti-mouse IgG were prepared by standard methods using the reactive succcinimidyl esters of the following fluorophores: Dye 3, Dye 14, Mr121, Alexa 660, Alexa 680, DyLight 680. A solution of the antibody was prepared at 20 mg/mL in 0.1 M phosphate buffer (pH=8.0). The labeling reagent was dissolved in DMSO or water at 10 mg/mL. Predetermined amounts of the labeling reagents were added to the protein solutions with stirring. A molar ratio of 10 equivalents of dye to 1 equivalent of protein was typical. The reaction mixture was incubated at room temperature for one hour. The dye-protein conjugates were separated on Sephadex G-25 column equilibrated with phosphate buffer. The fast running, protein-containing colored bands were collected and the degree of labeling (DOL) was determined from the absorbance at the absorbance maximum of each fluorophore. The absorbance of the dye at 280 nm was subtracted from the total absorbance of the conjugate at 280 nm to get the protein concentration.

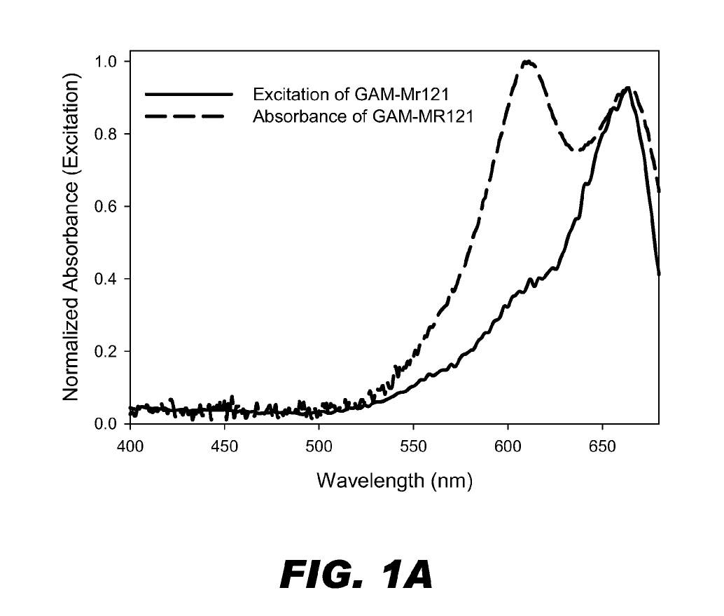

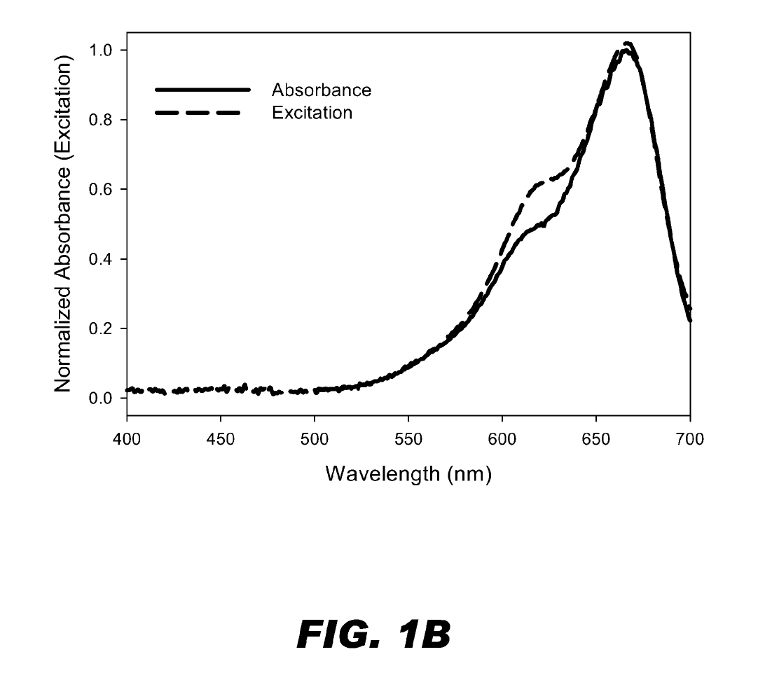

Comparison of the absorbance of a goat-anti mouse IgG conjugate of Dye 3 (DOL=5) and a goat anti-mouse IgG conjugate of oxazine dye Mr121 (DOL=4) at the same protein concentration is shown in FIG. 1. The conjugate of the present invention exhibits only one absorption peak at 667 nm, whereas the Mr121 conjugate exhibits two peaks (610 and 660 nm). The 610 nm Mr121 peak is due to the presence of nonfluorescent dye dimers, as this peak is absent in excitation spectra. The tendency of known oxazine dye Mr121 to aggregate limits the useful signal that can be obtained from the dye-protein conjugate.

### Example 32

### Total Fluorescence of Antibody Conjugates

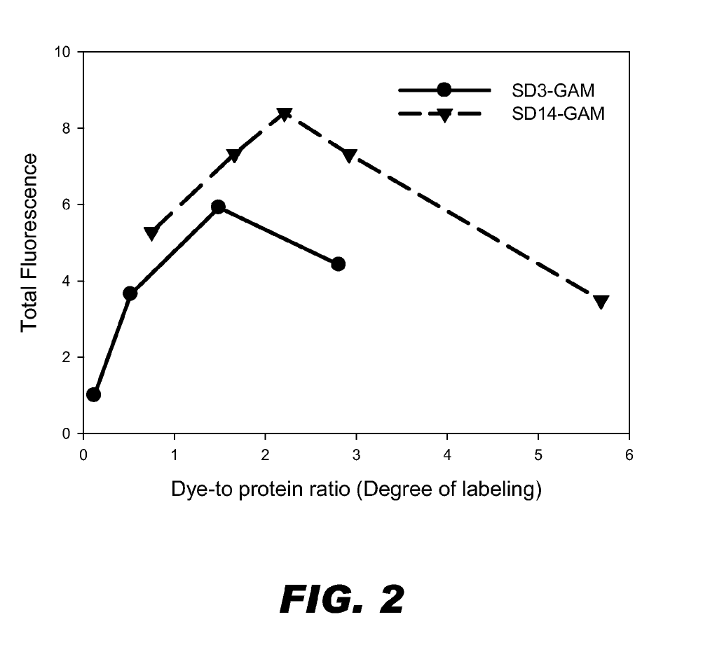

A series of goat anti-mouse IgG conjugates of Dye 3 and Dye 14 were prepared as in Example 31 to yield derivatives with different DOLs. The fluorescence of the conjugates was measured in fluorometer. As shown in FIG. 2, maximal fluorescence intensity was observed for conjugates with DOL 2.5.

### Example 33

### Photobleaching of Dye-Protein Conjugates

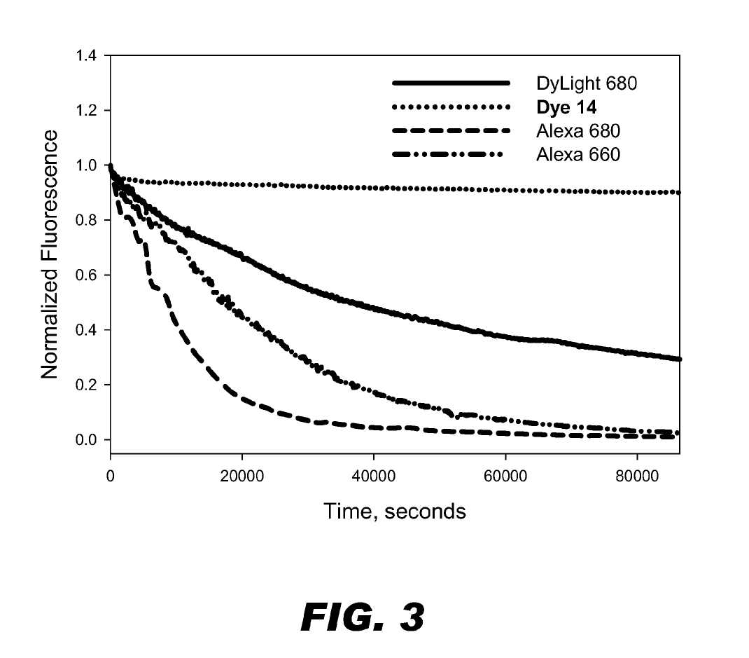

Antibody conjugates of Alexa 660, Alexa 680, DyLight 680, and Dye 14 were prepared with degrees of labeling of approximately 3-5. A solution of each conjugate (1 μM) was placed in a fluorometer cuvette. The fluorescence of the sample was monitored with constant light excitation at 650 nm. Fluorescence traces are shown in FIG. 3. Photobleaching followed first-order kinetics. Rate constants were obtained from a plot of In F vs time. As shown in Table 4, the antibody conjugates of the present invention are 25-150 fold more photostable than conjugates of other commercially available dyes with similar absorbance in near-infrared area 660-700 nm.

TABLE 4
Photobleaching of anti-mouse IgG labeled with Alexa 660,
Alexa 680, DyLight 680 and Dye 14.

| | Photobleaching | Relative photo- |
|---|---|---|
| Conjugate | rate (s⁻¹) | bleaching rate |
| IgG-Alexa 660 | 4.24 * 10⁻⁵ | 147 |
| IgG-Alexa 680 | 8.75 * 10⁻⁵ | 71 |
| IgG-DyLight 680 | 1.46 * 10⁻⁵ | 25 |
| IgG-Dye 14 | 8.75 * 10⁻⁷ | 1.00 |

### Example 34

### Fluorescence Quenching with Tryptophan

Fluorescence of oxazine dye Mr121 can be strongly quenched by the amino acid tryptophan. The quenching occurs via photoinduced electron transfer (PET) from a donor heterocycle to fluorophore singlet excited state following thermal back electron transfer to regenerate the fluorophore ground state. Such quenching is highly valuable for preparation of enzyme activity sensors and was utilized in preparation of protease biosensors (Sauer M., Angew. Chem. Int. Ed., 2004 (43)3798-3801).

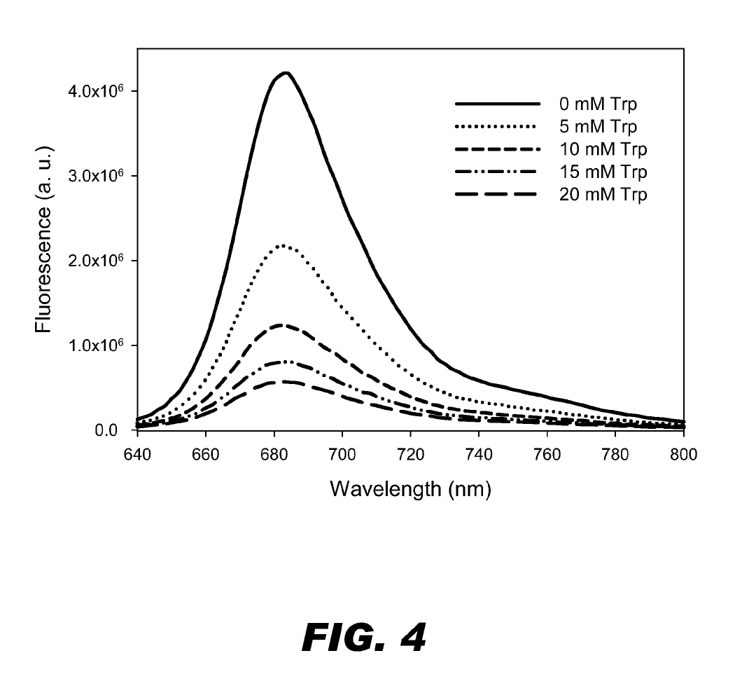

Steady-state fluorescence spectroscopy was performed for Dye 2, Dye 3, Dye 14, and Mr121 dye in presence of tryptophan at 25° C. in phosphate buffer. Typical fluorescence spectra of Dye 3 dye in the presence and absence of tryptophan are presented in FIG. 4.

For evaluating the quenching efficiency, the steady-state fluorescence quenching results were analyzed following the Stern-Volmer equation F₀/F=1+K_{Q}[Q], where Fₒ and F are the fluorescence intensities in the absence and presence of tryptophan and K_{Q} is a static Stern-Volmer constant. The calculated static Stern-Volmer constants are presented in Table 5.

TABLE 5
Fluorescence quenching data for the
oxazine dyes with tryptophan in phosphate
buffered solutions, pH = 8.0.

| | Tryptophan |
|---|---|
| | Stern-Volmer static |
| Dye | quenching constant, K_{Q} |
| Mr121 | 176 ± 12 |
| Dye 2 | 263 ± 24 |
| Dye 3 | 320 ± 26 |
| Dye 14 | 269 ± 22 |

The quenching constants for Dye 2, Dye 3 and Dye 14 are higher than the corresponding constant for Mr121. The larger constants will allow preparing more sensitive biosensors with greater fluorescence signals.

1. A compound comprising Formula (I): 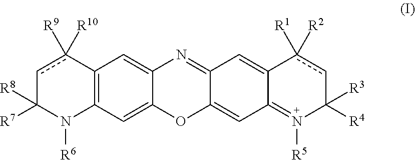
wherein:
R¹, R², R⁹, and R¹⁰ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, sulfonate, and amide;
R³, R⁴, R⁷, and R⁸ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide; provided that R³ and R⁴ together and/or R⁷ and R⁸ together may form a saturated and/or unsaturated bridge;
R⁵ and R⁶ are independently selected from the group consisting of {—}L₁-Rₓ, hydrogen, hydrocarbyl, substituted hydrocarbyl, and a group that has a positive charge or negative charge at a pH from about 6 to about 8; provided that at least one of R⁵ or R⁶ comprises a group that has a positive charge or negative charge at a pH from about 6 to about 8; provided that if one of R⁵ or R⁶ is an alkyl group then the other of R⁵ or R⁶ does not comprise a carboxylate group;
L₁ is a linker;
Rₓ is a group selected from the group consisting of phosphate, sulfonate, carboxylic acid, carboxylic ester, isocyanate, isothiocyanate, maleimide, haloacetamide, and alkyl halide; and
-------- is an optional double bond; provided that when a double bond is present then R² and/or R¹⁰ are not present; and provided that when R¹ is SO₃H and R² is hydrogen or not present, then at least one of R⁷ or R⁸ is not hydrogen.

2. The compound of claim 1, wherein the hydrocarbyl and/or substituted hydrocarbyl is selected from the group consisting of alkyl, cycloalkyl, alkenyl, alkenoxy, aryl, alkylaryl, arylalkoxyl, alkoxy, alkoxycarbonyl, carbonyl, acyl, acyloxy, sulfonyl, sulfonyl halide, sulfonyl ester, carboxyl, and carboxylic acid.

3. The compound of claim 2, wherein at least one of the carbon atoms forming the hydrocarbyl or substituted hydrocarbyl is substituted with a group selected from the group consisting of hydroxyl, halogen, cyano, nitro, carbonyl, acyl, acyloxy, carboxyl, phosphate, sulfonyl, sulfonyl halide, carboxylic acid ester, sulfonyl ester, amino, amido, azide, thiocyanate, isothiocyanate, and imide.

4. The compound of claim 1, wherein the group that has a positive charge or negative charge at a pH from about 6 to about 8 comprises {—}L₂-X, wherein L₂ is a linker attaching X to a nitrogen ring atom, and X is selected from the group consisting of sulfate, sulfonate, sulfinate, phosphate, phosphonate, phosphinate, carboxylate, amine, alkyl amine, dialkylamine, and an ammonium salt.

5. The compound of claim 4, wherein L₁ and L₂ are selected from a hydrocarbyl and a substituted hydrocarbyl.

6. The compound of claim 5, wherein L₁ and L₂ are selected from the group consisting of alkyl groups, alkenyl groups, aryl groups, and heteroaryl groups.

7. The compound of claim 1, wherein at least one of R¹, R², R³, R⁴, R⁵, R⁶, R⁷, R⁸, R⁹, and R¹⁰ is {—}L₁-Rₓ.

8. The compound of claim 1, wherein at least two of R¹, R², R³, R⁴, R⁵, R⁶, R⁷, R⁸, R⁹, and R¹⁰ are {—}L₁-Rₓ.

9. The compound of claim 1, wherein at least three of R¹, R², R³, R⁴, R⁵, R⁶, R⁷, R⁸, R⁹, and R¹⁰ are { }L₁-Rₓ.

10. The compound of claim 1, wherein R³, R⁴, R⁷, and R⁸ are independently selected from the group consisting of hydrogen and an alkyl group.

11. The compound of claim 1, wherein R³, R⁴, R⁷, and R⁸ are independently selected from the group consisting of hydrogen and methyl.

12-25. (canceled)

26. The compound of claim 1, wherein the optional double bonds are not present and one of R¹ or R² is SO₂X¹, wherein X¹ is selected from the group consisting of O⁻, ONa, OLi, OK, ONH₄, and ONR₄, wherein R is an alkyl group.

27. (canceled)

28. The compound of claim 26,
wherein:
R⁵ and R⁶ are independently selected from the group consisting of {—}(CH₂)ₘCOX² and {—}(CH₂)ₘSO₃⁻;
m is an integer from 1 to 5;
X² is selected from the group consisting of hydroxyl, a N-succinimidyl ester group, and a 2-(N-maleimidoethyl)amino group; and
one of R⁹ and R¹⁰ is hydrogen, and the other of R⁹ and R¹⁰ is selected from the group consisting of hydrogen, methyl, and
{—}(CH₂)ₘSO₃⁻.

29-42. (canceled)

43. The compound of claim 1, wherein both optional double bonds are present and one of R¹ or R⁹ is SO₂X¹, wherein X¹ is selected from the group consisting of O⁻, ONa, OLi, OK, ONH₄, and ONR₄, wherein R is an alkyl group.

44. (canceled)

45. The compound of claim 43, wherein R⁵ and R⁶ are independently selected from the group consisting of {—}(CH₂)ₘCOX² and {—}(CH₂)ₘSO₃⁻, wherein m is an integer from 1 to 5; and X² is selected from the group consisting of hydroxyl, a N-succinimidyl ester group, and a 2-(N-maleimidoethyl)amino group.

46-60. (canceled)

61. The compound of claim 28, wherein R³, R⁴, R⁷, and R⁸ are independently selected from the group consisting of hydrogen and an alkyl group.

62. The compound of claim 61, wherein R³, R⁴, R⁷, and R⁸ are independently selected from the group consisting of hydrogen and methyl.

63. The compound of claim 61, wherein R³, R⁴, R⁷, and R⁸ are each methyl.

64. The compound of claim 61, wherein R³, and R⁴ are each methyl; and R⁷, and R⁸ are each hydrogen.

65. The compound of claim 61, wherein R³, and R⁴ are each hydrogen; and R⁷, and R⁸ are each methyl.

66-80. (canceled)

81. The compound of claim 45, wherein R³, R⁴, R⁷, and R⁸ are independently selected from the group consisting of hydrogen and an alkyl group.

82. The compound of claim 81, wherein R³, R⁴, R⁷, and R⁸ are independently selected from the group consisting of hydrogen and methyl.

83. The compound of claim 81, wherein R³, R⁴, R⁷, and R⁸ are each methyl.

84. The compound of claim 81, wherein R³, and R⁴ are each methyl; and R⁷, and R⁸ are each hydrogen.

85. The compound of claim 81, wherein R³, and R⁴ are each hydrogen; and R⁷, and R⁸ are each methyl.

86-88. (canceled)

89. A process for preparing a compound comprising Formula (II): 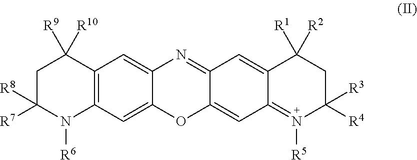
the process comprising a step selected from the group consisting of:
(a) contacting a compound comprising Formula (i) with a compound comprising Formula (ii) in the presence of a proton donor to form the compound comprising Formula (II): 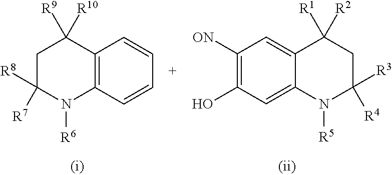
(b) contacting a compound comprising Formula (iii) with a compound comprising Formula (iv) in the presence of a proton donor to form the compound comprising Formula (II); and 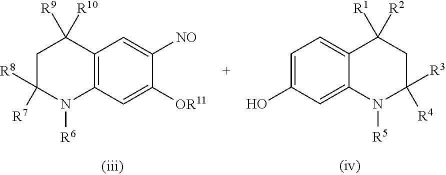
(c) contacting a compound comprising Formula (v) with a compound comprising Formula (iv) in the presence of a proton donor to form the compound comprising Formula (II): 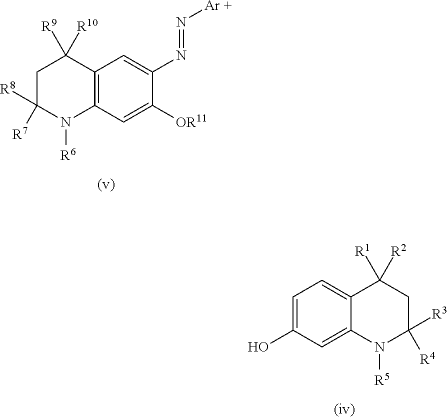
wherein:
R¹, R², R⁹, and R¹⁰ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, sulfonate, and amide;
R³, R⁴, R⁷, and R⁸ are independently selected from the group consisting of {—}L₁-Rₓ, hydrocarbyl, substituted hydrocarbyl, hydrogen, hydroxyl, halogen, cyano, nitro, phosphate, amino, amido, azide, thiocyanate, isothiocyanate, and amide; provided that R³ and R⁴ together and/or R⁷ and R⁸ together may form a saturated and/or unsaturated bridge;
R⁵ and R⁶ are independently selected from the group consisting of {—}L₁-Rₓ, hydrogen, hydrocarbyl, substituted hydrocarbyl, and a group that has a positive charge or negative charge at a pH from about 6 to about 8; provided that at least one of R⁵ or R⁶ comprises a group that has a positive charge or negative charge at a pH from about 6 to about 8; provided that if one of R⁵ or R⁶ is an alkyl group then the other of R⁵ or R⁶ does not comprise a carboxylate group;
R¹¹ is selected from the group consisting of hydrogen and alkyl;
Ar comprises C₆H₄Xₐ;
Xₐ comprises an electron withdrawing group; and
the proton donor has a pKa less than about 6.
